(19) United States
(12) Patent Application Publication
(10) Pub. No.: US 2025/0228825 A1
(43) Pub. Date: Jul. 17, 2025

(54) COMPOSITIONS OF RAPAMYCIN AND THEIR USE IN THE TREATMENT OF MICROCYSTIC LYMPHATIC MALFORMATION

(71) Applicant: PALVELLA THERAPEUTICS, INC., Wayne, PA (US)

(72) Inventor: Jeffrey MARTINI, Malvern, PA (US)

(73) Assignee: PALVELLA THERAPEUTICS, INC., Wayne, PA (US)

(21) Appl. No.: 18/690,921

(22) PCT Filed: Sep. 15, 2022
(86) PCT No.: PCT/US2022/043627

§ 371 Date: Mar. 11, 2024

Related U.S. Application Data
(60) Provisional application No. 63/244,391, filed on Sep. 15, 2021.

(51) Int. Cl. A61K 31/436; A61K 9/00; A61K 9/06; A61K 47/10; A61K 47/12; A61K 47/14; A61K 47/22; A61K 47/38; A61P 17/02

(52) U.S. Cl. CPC A61K 31/436; A61K 9/0014; A61K 9/06; A61K 47/10; A61K 47/12; A61K 47/14; A61K 47/22; A61K 47/38; A61P 17/02

(57) ABSTRACT

Embodiments herein are directed to methods and compositions for treating microcystic lymphatic malformation and more specifically to topical rapamycin gels and methods of their use. Methods may be effective to reduce the size, severity, or number of lesions and may improve quality of life and ability to function for a subject suffering from microcystic lymphatic malformations.

### CROSS-REFERENCE TO RELATED APPLICATIONS

This application claims the benefit under 35 U.S.C. § 119 (e) to U.S. Application Ser. No. 63/244,391 filed Sep. 15, 2021. The disclosure of each of the prior applications is considered part of and is incorporated by reference in the disclosure of this application.

### BACKGROUND

Lymphatic malformation (LM) is a rare disease of the lymphatic system characterized by abnormal vessels, outpouchings, or cysts filled with lymphatic fluid. According to the International Society for the Study of Vascular Anomalies (ISSVA), microcystic LM is one of three morphologic types of LMs based on the size of the individual cysts (as opposed to the overall size of the LM): macrocystic (>2 cm), microcystic (<2 cm), and combined.

The manifestations of LM differ in their presentation and anatomical location (i.e., skin for microcystic or deeper tissues for macrocystic) but are always the result of diffuse infiltration and overgrowth of localized masses. Unlike macrocystic LM, microcystic LMs permeate the subcutaneous tissue with clinical presentation on the skin. Microcystic LM can occur in any anatomic region of the body that has a lymphatic network. Microcystic LM contain multiple microcysts that together form the lesion. The actual size of the LM lesion varies, depending on the total number of individual microcysts.

Microcystic LM presents at birth and is the result of congenital abnormalities of the lymphatic system thought to originate during the embryologic development of lymphatic vessels. All LMs arise due to post zygotic mutations during early embryonic development that that lead to malformed lymphatic vasculature, persistent infiltration of lymph fluid into soft tissues and locally invasive masses with pathologic sequelae. Microcystic LM affects men and women of equal proportions though some LM is influenced by hormonal changes at puberty. The natural history of microcystic LM is progressive, worsening during life with increases in the number of cysts leading to an increase in overall size, complications, and morbidity.

The natural history of microcystic LM is progressive worsening during life with increases in lesion size, number, complications, and morbidity. Microcystic LM, often referred to as LM lesions, infiltrate the skin resulting in spontaneous leakage of lymph fluid and blood from pathologic vesicles. Bacteria can enter through these LM lesions, and quickly spread through affected tissues, resulting in recurrent, severe infection. This substantially impairs quality of life by limiting function and can result in life-threatening consequences such as sepsis. Furthermore, if there is substantial associated bleeding, these lesions can also result in systemic anemia.

Based on the need for effective therapies to treat microcystic LM, this Phase 2 study was designed to evaluate the safety, PK, and efficacy of PTX-022 in participants with microcystic LM. The study will evaluate safety, including determining sirolimus plasma concentration. Efficacy will be evaluated using patient and clinician assessments. Since microcystic LM rarely regress, each participant will serve as their own control for this open label study.

The natural history of microcystic LM is progressive worsening during life with increases in size, number, complications, and morbidity. Microcystic LM can occur in any anatomic region of the body that has a lymphatic network, but around 48% occur in the head and neck.

Affected patients suffer from a wide array of complications related to their microcystic LM (also referred to as LM lesion), which are associated with a high rate of persistent morbidity. Complications include bleeding, leaking (lymphorrhea), pain, itching, functional impairment, infection, and disfigurement. The bleeding and leaking lead to the LM lesions becoming swollen, painful, and recurrently infected, potentially leading to dissemination. In addition, LM lesions can substantially impair quality of life and persistently limit function.

LM arise due to mutations in vascular and lymphatic formation during early embryonic development with abnormal vascular and lymphatic vessel makeup and connectivity. They are congenital, sporadic, and arise from somatic germline mutations and are usually present and diagnosed at birth. Some LM become exasperated by hormonal changes at puberty. They affect men and women of equal proportions.

Effective management of LM requires cross-disciplinary care. Oftentimes, imaging with computed tomography or magnetic resonance imaging is undertaken prior to initiation of therapy, in order to help with treatment selection and prognostication. Unfortunately, many available therapeutic options (sclerotherapy, laser therapy, and surgery) are invasive, painful, and can induce further inflammation/scarring. Furthermore, the efficacy of these treatments, especially for microcytic LM, is typically incomplete and transient, with high recurrence rates. Thus, many patients with microcystic LM continue to experience lifelong clinical and psychosocial morbidity, and in many cases have deteriorating quality of life over time. While there are no FDA-approved medications for microcystic LM, many recommend oral sirolimus as a first-line therapy for this highly morbid condition.

Despite the serious, lethal consequences of microcystic LM, there are no FDA-approved medications for this disease. In fact, currently available treatment options (surgery, sclerotherapy, laser, cryotherapy) are invasive, can induce further scarring/inflammation, and are not completely efficacious, resulting in high recurrence rates. Ultimately, therefore, there is a critical need for a therapy that can address the root cause of these aberrant lymphatic formations.

### SUMMARY OF THE INVENTION

In one embodiment, the present invention provides a method of treating microcystic lymphatic malformation in a subject in need thereof using topically administering once daily to the microcystic lymphatic malformation skin lesions of the subject a therapeutically effective amount of an anhydrous rapamycin gel composition wherein the microcystic lymphatic malformation is treated.

In one embodiment, the microcystic lymphatic malformation skin lesion is greater than about 25 cm². In one embodiment, a symptom related to the microcystic lymphatic malformation skin lesion occurs at least 1-2 days per week. In one embodiment, treating microcystic lymphatic malformation in a subject is improving a symptom of the microcystic lymphatic malformation skin lesion is leaking (lymphorrhea), bleeding, redness, crusting, hyperkeratosis, elevated plaques, or combinations thereof. In one embodiment, the anhydrous rapamycin gel composition comprises about 0.1% to about 6% of rapamycin, about 80% to about 99% of one or more solvents, a gelling agent, and an antioxidant. In one embodiment, the anhydrous rapamycin gel composition comprises about 0.1% to about 6% of rapamycin, about 80% to about 99% of one or more solvents, about 0.1% to about 5% of a gelling agent, and about 0.001% to about 1% of an antioxidant. In one embodiment, the one or more solvents is diisopropyl adipate, glycerol, PEG, or isopropyl alcohol. In one embodiment, the anhydrous rapamycin gel composition has about 3.9% of rapamycin, about 15% isopropyl alcohol, about 54.9% polyethylene glycol 400, about 15% diisopropyl adipate, about 10% glycerol, about 0.75% hydroxypropyl cellulose, about 0.05% propyl gallate, about 0.02% ascorbyl palmitate, about 0.002% alpha-tocopherol, and citric acid.

In one embodiment, administering comprises applying the anhydrous rapamycin gel composition as a thin layer to the subject's skin. In one embodiment, the anhydrous rapamycin gel composition is administered once daily for at least 12 weeks.

In one embodiment, the subject shows improvement in a score on one or more of assessments selected from the group consisting of Patient Global Impression of Change (PGI-C), Patient Global Impression of Severity (PGI-S), Patient Global Impression of Severity (PGI-SL) for leaking, Patient Global Impression of Severity (PGI-SB) for bleeding, Clinician Global Impression of Change (CGI-C), Clinician Global Impression of Severity (CGI-S), and combinations thereof. In one embodiment, the subject shows improvement in a score on the dermatology life quality index (DLQI). In one embodiment, the subject experiences a decrease in the number of days in which lesion leaking is reported. In one embodiment, the subject experiences a decrease in the number of days in which lesion bleeding is reported. In one embodiment, one or more of lesion size lesion thickness, lesion height, or lesion number is reduced. In one embodiment, the severity of lesion pain, lesion crusting or lesion redness based on the Microcystic LM Symptom Severity (SS) Scale is reduced. In one embodiment, field radiation (XRT) administered to the mycrocystic lymphatic malformation skin lesion is not necessary.

In one embodiment, topical administration of the anhydrous rapamycin gel composition to the skin of a subject achieves a maximum epidermis concentration of rapamycin in the epidermis of the subject of about 120-990 micromolar rapamycin. In one embodiment, topical administration of the anhydrous rapamycin gel composition to the skin of a subject achieves a maximum dermis concentration of rapamycin in the dermis of the subject of about 50-200 micromolar rapamycin.

### BRIEF DESCRIPTION OF THE DRAWINGS

FIG. 1 is a graph showing the Clinician Global Impression of Change (CGI-C) for subjects 1-10.

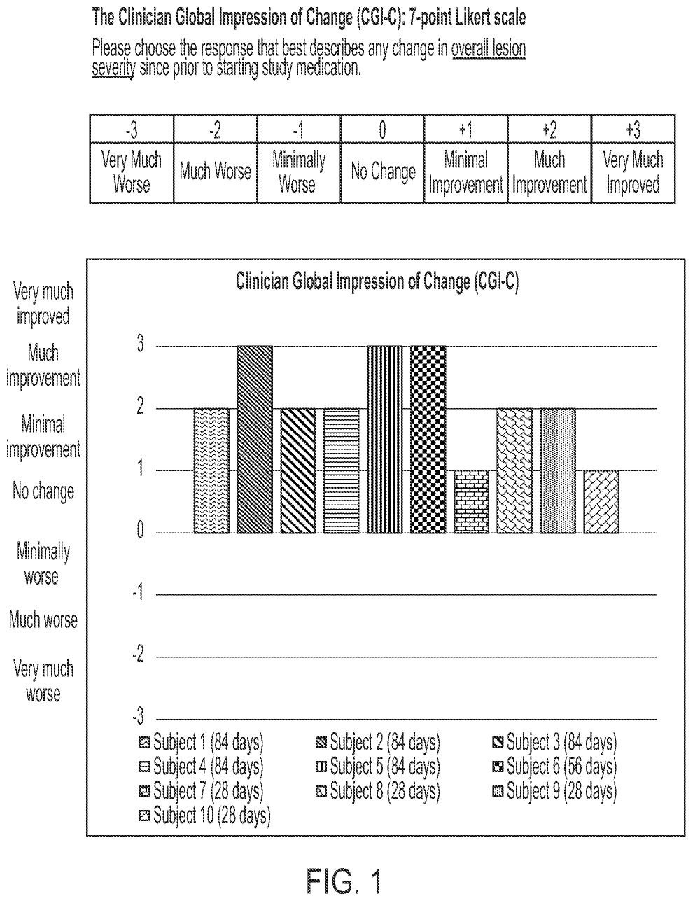

FIG. 2 is a graph showing the Change Clinician Global Impression of Severity (CGI-C) for crusting/hyperkeratosis, erythema, height, bleeding, leaking for subjects 1-10.

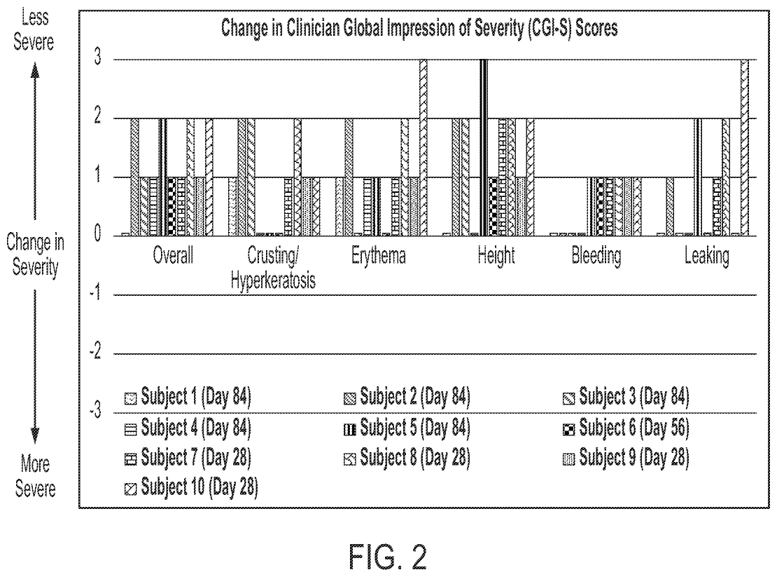

FIG. 3 is a graph showing the Patient Global Impression of Change (PGI-C) Scores for subjects 1-10.

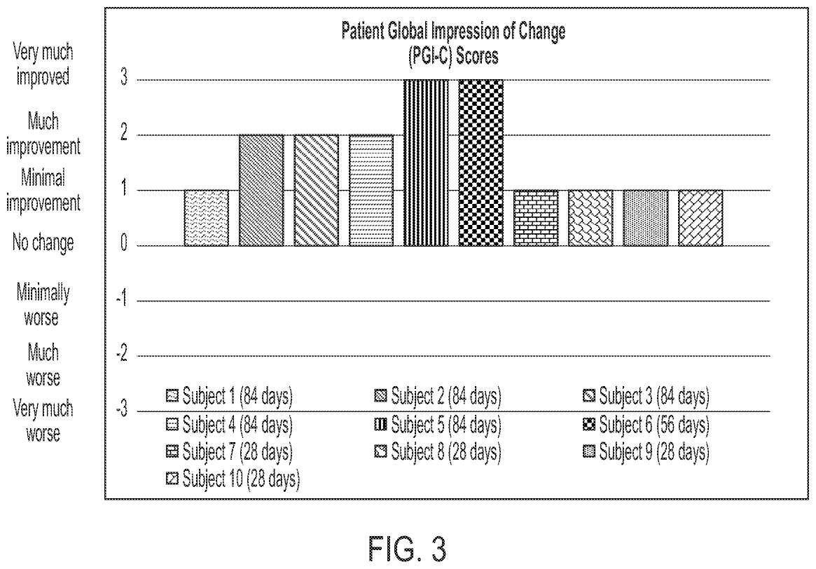

FIG. 4 shows the evaluation of microcystic LM for subject 1 at baseline, day 28, day 56 and day 84.

FIG. 5 shows the evaluation of microcystic LM for subject 2 at baseline, day 28, day 56 and day 84.

FIG. 6 shows the evaluation of microcystic LM for subject 3 at baseline, day 28 and day

56.

FIG. 7 shows the evaluation of microcystic LM for subject 5 at baseline, day 28, day 56 and day 84.

FIG. 8 shows the evaluation of microcystic LM for subject 6 at baseline, day 28 and day 56.

FIG. 9 shows the evaluation of microcystic LM for subject 7 at baseline and day 28.

FIG. 10 shows the evaluation of microcystic LM for subject 8 at baseline and day 28.

FIG. 11 shows the evaluation of microcystic LM for subject 9 at baseline and day 28.

FIG. 12 shows the evaluation of microcystic LM for subject 10 at baseline and day 28.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### Methods of Use

Disclosed herein are methods of treating microcystic lymphatic malformations (microcystic LM) in a subject. In particular, anhydrous rapamycin gel compositions are provided that may decrease one or more of the symptoms of microcystic LM, including lymphorrhea frequency, bleeding frequency, lesion severity, lesion size, lesion height, number of lesions, pruritus severity and frequency, and pain severity and frequency.

Embodiments described herein are directed to methods of treating lymphorrhea frequency and bleeding frequency in a subject in need thereof comprising topically administering once daily to the microcystic LM skin lesions of the subject a therapeutically effective amount of an anhydrous rapamycin gel composition wherein the microcystic LM is treated.

In certain embodiments, the microcystic LM skin lesion is greater than about 25 cm².

In certain embodiments, a symptom related to the microcystic LM skin lesion occurs at least 1-2 days per week or ≥5 days over the past 14 days. In certain embodiments, the symptom related to the microcystic LM skin lesion is selected from the group consisting of leaking, bleeding, redness, crusting, hyperkeratosis, elevated plaques, and combinations thereof.

As such, in one aspect, the present disclosure provides a method of treating microcystic LM in a subject in need thereof comprising topically administering once daily to the microcystic LM skin lesions of the subject a therapeutically effective amount of an anhydrous rapamycin gel composition comprising about 0.1 wt. % to about 6 wt. % of rapamycin, about 80 wt. % to about 99 wt. % of one or more solvents, a gelling agent, and an antioxidant. In any embodiment, the anhydrous rapamycin gel composition comprises about 0.1 wt. % to about 6 wt. % of rapamycin, about 80 wt. % to about 99 wt. % of one or more solvents, about 0.1 wt. % to about 5 wt. % of a gelling agent, and about 0.001 wt. % to about 1 wt. % of an antioxidant. For example, a method of treating microcystic LM in a subject in need thereof comprises topically administering once daily to the microcystic LM skin lesions of the subject a therapeutically effective amount of an anhydrous rapamycin gel composition comprising about 0.1 wt. % to about 6 wt. % of rapamycin, about 5 wt. % to about 30 wt. % diisopropyl adipate, about 5 wt. % to about 30 wt. % glycerol, about 40 wt. % to about 60 wt. % polyethylene glycol (PEG), about 5 wt. % to about 30 wt. % isopropyl alcohol, a gelling agent, and optionally an antioxidant (such as citric acid).

In preferred embodiments, methods of treating microcystic LM in a subject in need thereof comprises topically administering once daily to the microcystic LM skin lesions of the subject a therapeutically effective amount of an anhydrous rapamycin gel composition comprising about 3.9 wt. % of rapamycin, about 15 wt. % diisopropyl adipate, about 10% glycerol, about 54.9 wt. % polyethylene glycol (PEG), about 15 wt. % isopropyl alcohol, about 0.75% hydroxypropyl cellulose, about 0.05% propyl gallate, about 0.02% ascorbyl palmitate, about 0.002% alpha-tocopherol, and citric acid.

As used herein, “rapamycin” or “sirolimus” are used interchangeably and refer to the macrocyclic lactone produced by the organism Streptomyces hydroscopicus isolated from soil samples of Easter Island (Rapa Nui) and having the structure of:

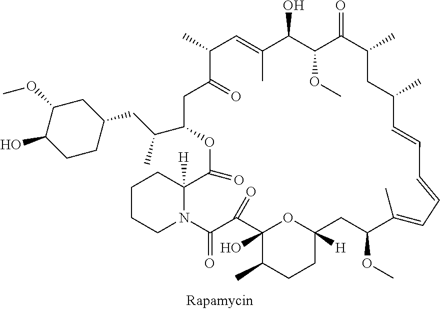 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

An anhydrous rapamycin gel composition suitable for use in the treatment of microcystic LM, as disclosed herein, may be administered topically by any methods disclosed herein. For example, administration may comprise topically applying an anhydrous rapamycin gel composition, as disclosed herein, as a thin layer over one or more of an microcystic LM skin lesion, wherein the skin lesion is anywhere on the body including the chest, the neck, the scalp, the back, or the face. Topical administration of an anhydrous rapamycin gel composition, as disclosed herein, may result in rapamycin reaching one or both of the epidermal layer and dermal layer of the skin through absorption. Advantageously, topical administration of an anhydrous rapamycin gel composition, as disclosed herein, may result in no or minimal systemic absorption of rapamycin.

Topical administration of an anhydrous rapamycin gel composition, as disclosed herein, is administered in the evening, at least one hour before bed. The anhydrous rapamycin gel composition, disclosed herein, should be applied once daily to the microcystic LM skin lesion. Topical administration of an anhydrous rapamycin gel composition, as disclosed herein, may be carried out once daily, twice daily, or three times daily. Alternatively, an anhydrous rapamycin gel composition, as disclosed herein, may be topically administered 3 times per week, 2 times per week, 1 time per week, or once every other day.

Topical administration of an anhydrous rapamycin gel composition as disclosed herein may be administered for a period of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or preferably, at least 12 weeks. More preferably, topical administration of an anhydrous rapamycin gel composition, as disclosed herein, may be administered for a period of at least 24 weeks, at least 3 months, at least 4 months, at least 5 months, at least 6 months, at least 8 months, at least 12 months, or longer.

Topical administration of an anhydrous rapamycin gel composition disclosed herein may be carried out in dosing cycles, wherein an anhydrous rapamycin gel composition is administered for a period of time, as described herein, administration ceases for a period of time, and then the administration resumes again for another period of time, as described herein. The time of cessation of the administration of an anhydrous rapamycin gel composition, as disclosed herein, may be about 1 week to about 12 weeks.

In any embodiment, topical administration of an anhydrous rapamycin gel composition to the affected skin of a subject with microcystic LM decreases lesion thickness, lesion height, or lesion size on the skin of the subject. In embodiments described herein, topical administration of an anhydrous rapamycin gel composition to the affected skin of a subject with microcystic LM decreases the mycrocystic lymphatic malformation skin lesion to less than about 25 cm².

In any embodiment, topical administration of an anhydrous rapamycin gel composition to the skin of a subject with microcystic LM decreases the number of lesions on the skin of the subject.

In any embodiment, topical administration of an anhydrous rapamycin gel composition to the skin of a subject with microcystic LM decreases the total surface area covered by lesions on the skin of the subject. In embodiments described herein, topical administration of an anhydrous rapamycin gel composition to the affected skin of a subject with microcystic LM decreases the total surface area covered by lesions to less than about 200 cm².

In any embodiment, topical administration of an anhydrous rapamycin gel composition to the skin of a subject with microcystic LM decreases lesion bleeding on the skin of the subject. In any embodiment, lesion bleeding is decreased to less than 7 days per week, less than 6 days per week, less than 5 days per week, less than 4 days per week, less than 3 days per week, less than 2 days per week, less than 1 day per week, or lesion bleeding has stopped. In any embodiment, lesion bleeding is decreased to less than 13 days over a 14 day period, less than 12 days over a 14 day period, less than 11 days over a 14 day period, less than 10 days over a 14 day period, less than 9 days over a 14 day period, less than 8 days over a 14 day period, less than 7 days over a 14 day period, less than 6 days over a 14 day period, less than 5 days over a 14 day period, less than 4 days over a 14 day period, less than 3 days over a 14 day period, less than 2 days over a 14 day period, less than 1 days over a 14 day period, or lesion bleeding has stopped.

In any embodiment, topical administration of an anhydrous rapamycin gel composition to the skin of a subject with microcystic LM decreases lesion lymphorrhea, leaking or oozing on the skin of the subject. In any embodiment, lesion leaking or oozing is decreased to less than 7 days per week, less than 6 days per week, less than 5 days per week, less than 4 days per week, less than 3 days per week, less than 2 days per week, less than 1 day per week, or lesion leaking or oozing has stopped. In any embodiment, lesion leaking or oozing is decreased to less than 13 days over a 14 day period, less than 12 days over a 14 day period, less than 11 days over a 14 day period, less than 10 days over a 14 day period, less than 9 days over a 14 day period, less than 8 days over a 14 day period, less than 7 days over a 14 day period, less than 6 days over a 14 day period, less than 5 days over a 14 day period, less than 4 days over a 14 day period, less than 3 days over a 14 day period, less than 2 days over a 14 day period, less than 1 days over a 14 day period, or lesion leaking or oozing has stopped.

In any embodiment, topical administration of an anhydrous rapamycin gel composition to the skin of a subject with microcystic LM decreases pruritus of the skin of the subject. In any embodiment, pruritus is decreased to less than 7 days per week, less than 6 days per week, less than 5 days per week, less than 4 days per week, less than 3 days per week, less than 2 days per week, less than 1 day per week, or pruritus has stopped. In any embodiment, pruritus is decreased to less than 13 days over a 14 day period, less than 12 days over a 14 day period, less than 11 days over a 14 day period, less than 10 days over a 14 day period, less than 9 days over a 14 day period, less than 8 days over a 14 day period, less than 7 days over a 14 day period, less than 6 days over a 14 day period, less than 5 days over a 14 day period, less than 4 days over a 14 day period, less than 3 days over a 14 day period, less than 2 days over a 14 day period, less than 1 days over a 14 day period, or pruritus has stopped.

In any embodiment, topical administration of an anhydrous rapamycin gel composition to the skin of a subject with microcystic LM decreases redness (or lesion color) of the skin of the subject. In any embodiment, redness is decreased to less than 7 days per week, less than 6 days per week, less than 5 days per week, less than 4 days per week, less than 3 days per week, less than 2 days per week, less than 1 day per week, or redness is gone. In any embodiment, redness is decreased to less than 13 days over a 14 day period, less than 12 days over a 14 day period, less than 11 days over a 14 day period, less than 10 days over a 14 day period, less than 9 days over a 14 day period, less than 8 days over a 14 day period, less than 7 days over a 14 day period, less than 6 days over a 14 day period, less than 5 days over a 14 day period, less than 4 days over a 14 day period, less than 3 days over a 14 day period, less than 2 days over a 14 day period, less than 1 days over a 14 day period, or redness has stopped.

In any embodiment, topical administration of an anhydrous rapamycin gel composition to the skin of a subject with microcystic LM decreases crusting of the skin of the subject. In any embodiment, crusting is decreased to less than 7 days per week, less than 6 days per week, less than 5 days per week, less than 4 days per week, less than 3 days per week, less than 2 days per week, less than 1 day per week, or crusting is gone. In any embodiment, crusting is decreased to less than 13 days over a 14 day period, less than 12 days over a 14 day period, less than 11 days over a 14 day period, less than 10 days over a 14 day period, less than 9 days over a 14 day period, less than 8 days over a 14 day period, less than 7 days over a 14 day period, less than 6 days over a 14 day period, less than 5 days over a 14 day period, less than 4 days over a 14 day period, less than 3 days over a 14 day period, less than 2 days over a 14 day period, less than 1 days over a 14 day period, or crusting has stopped.

In any embodiment, topical administration of an anhydrous rapamycin gel composition to the skin of a subject with microcystic LM decreases hyperkeratosis of the skin of the subject. In any embodiment, thickness of the lesion is reduced. In any embodiment, elevated plaques have been reduced.

In any embodiment, topical administration of an anhydrous rapamycin gel composition to the skin of a subject with microcystic LM results in the elimination of any additional therapies for the treatment of microcystic LM, such therapies include field radiation (XRT), sclera therapy, or laser therapy.

In any embodiment, topical administration of an anhydrous rapamycin gel composition to the skin of a subject with microcystic LM decreases pain or redness of the skin of the subject, which may be measured, using a 0-10-point scale ranking pain, and redness from a scale of 0 (no symptom) to 10 (severe). For example, a subject may see an improvement of at least 1 point, at least 2 points, at least 3 points, at least 4 points, at least 5 points, at least 6 points, at least 7 points, at least 8 points, or at least 9 points in the relevant score after topically administering once daily an anhydrous rapamycin gel composition, as disclosed herein.

In any embodiment, the improvement in symptoms of microcystic LM may be reported by a subject's clinician, for example, through Clinician Global Impression of Severity (CGI-S), which is a global index that may be used to rate a clinician's perception of severity of a specific condition or the Clinician Global Impression of Change (CGI-C), which reflects a clinician's belief about the efficacy of treatment. For example, a subject may see an improvement of at least 1 point, at least 2 points, at least 3 points, at least 4 points, or 5 points in the score on the CGI-S after topically administering once daily an anhydrous rapamycin gel composition, as disclosed herein. Similarly, a subject may see an improvement of at least 1 point, at least 2 points, at least 3 points, at least 4 points, at least 5 points, or 6 points in the score on the CGI-C after topically administering once daily an anhydrous rapamycin gel composition, as disclosed herein.

In any embodiment, the improvement in symptoms of microcystic LM may be reported by the subject themselves, for example through the dermatology life quality index (DLQI). For example, a subject may see an improvement of at least 2 points, at least 3 points, at least 4 points, at least 5 points, at least 6 points, at least 7 points, at least 8 points, at least 9 points, at least 10 points, at least 15 points, at least 20 points, at least 25 points, or at least 30 points in the score on the DLQI after topically administering once daily an anhydrous rapamycin gel composition, as disclosed herein.

In any embodiment, the improvement in symptoms of microcystic LM may be reported by the subject themselves, for example through the Patient Global Impression of Severity (PGI-S), which is a global index that may be used to rate patient's perception of severity of a specific condition and ranks severity on a scale of 0 (none) to 4 (very severe). For example, a subject may see an improvement of at least 1 point, at least 2 points, at least 3 points, or 4 points in the score on the PGI-S after topically administering once daily an anhydrous rapamycin gel composition, as disclosed herein. The PGI-S can be used to assess severity of particular symptoms, such as lesion bleeding (PGI-SB) and/or leaking (PGI-SL). As such, in any embodiment, a subject may see an improvement of at least 1 point, at least 2 points, at least 3 points, or 4 points in the score on the PGI-SB with respect to lesion bleeding after topically administering once daily an anhydrous rapamycin gel composition, as disclosed herein. Similarly, a subject may see an improvement of at least 1 point, at least 2 points, at least 3 points, or 4 points in the score on the PGI-SL with respect to lesion leaking after topically administering once daily an anhydrous rapamycin gel composition, as disclosed herein.

In any embodiment, the improvement in symptoms of microcystic LM may be reported by the subject themselves, for example through the Patient Global Impression of Change (PGI-C), which reflects a patient's belief about the efficacy of treatment on a scale of −3 (very much worse) to 3 (very much improved). For example, a subject may see an improvement of at least 1 point, at least 2 points, at least 3 points, at least 4 points, at least 5 points, or 6 points in the score on the PGI-C after topically administering once daily an anhydrous rapamycin gel composition, as disclosed herein.

In any embodiment, a subject may see an overall improvement in the ability to function, such increasing the number of days where the subject is able to perform day-to-day activities with little interference from lesion bleeding or leaking, after topically administering once daily an anhydrous rapamycin gel composition, as disclosed herein.

### Topical Compositions

An anhydrous rapamycin gel composition suitable for use in the treatment of microcystic LM, as disclosed herein, may comprise about 0.1 wt. % to about 6 wt. % of rapamycin, about 80 wt. % to about 99 wt. % of one or more solvents, about 0.1 wt. % to about 5 wt. % of a gelling agent, about 0.001 wt. % to about 1 wt. % of an antioxidant, and optionally a buffer.

In any embodiment, an anhydrous rapamycin gel composition may comprise about 0.1 wt. % to about 5 wt. %, about 0.1 wt. % to about 4.5 wt. %, about 0.1 wt. % to about 4 wt. %, about 0.1 wt. % to about 3.5 wt. %, about 0.1 wt. % to about 3 wt. %, about 0.1 wt. % to about 2.5 wt. %, about 0.1 wt. % to about 2 wt. %, about 0.1 wt. % to about 1.5 wt. %. For example, an anhydrous rapamycin gel composition disclosed herein may comprises rapamycin in an amount of about 0.1 wt. % to about 1 wt. %, about 1 wt. % to about 5 wt. %, about 1.5 wt. % to about 5 wt. %, about 2 wt. % to about 5 wt. %, about 2.5 wt. % to about 5 wt. %, about 3 wt. % to about 5 wt. %, about 3.5 wt. % to about 5 wt. %, about 4 wt. % to about 5 wt. %, about 4.5 wt. % to about 5 wt. %. Preferably, an anhydrous rapamycin gel composition disclosed herein comprises about 3.9 wt. % rapamycin.

### Solvent System

An anhydrous rapamycin gel composition suitable for use in the treatment of microcystic LM, as disclosed herein, comprises one or more solvents in an amount of about 80 wt. % to about 99 wt. %, about 80 wt. % to about 98 wt. %, about 80 wt. % to about 97 wt. %, about 80 wt. % to about 96 wt. %, about 80 wt. % to about 95 wt. %, about 80 wt. % to about 90 wt. %, about 80 wt. % to about 85 wt. %. Preferably, an anhydrous rapamycin gel composition suitable for use in the treatment of microcystic LM, as disclosed herein, comprises about 95 wt. % of one or more solvents. More preferably, an anhydrous rapamycin gel composition suitable for use in the treatment of microcystic LM, as disclosed herein, comprises four solvents present in a total amount of about 94.9 wt. %.

The one or more solvents suitable for use in an anhydrous rapamycin gel composition suitable for use in the treatment of microcystic LM, as disclosed herein, may each independently be selected from propylene glycol, benzyl alcohol, DMSO, diglycol, propylene glycol monocaprylate, diethylene glycol monoethylether, tetrahydrofurfurylalcohol polyethylene glycol ether, butylene glycol, diethylene glycol, triethylene glycol, polyethylene glycol, diisopropyl adipate, isopropyl alcohol, glycerol, and combinations thereof. Preferably, an anhydrous rapamycin gel composition suitable for use in the treatment of microcystic LM, as disclosed herein, may comprise four solvents comprising isopropyl alcohol, polyethylene glycol 400, diisopropyl adipate, and glycerol.

In any embodiment, an anhydrous rapamycin gel composition suitable for use in the treatment of microcystic LM, as disclosed herein, may comprise isopropyl alcohol as one of the one or more solvents in an amount of about 5 wt. % to about 55 wt. %. In any embodiment, an anhydrous rapamycin gel composition may comprise isopropyl alcohol as the one or more solvents in an amount of about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, or about 5 wt. % to about 10 wt. %. Preferably, an anhydrous rapamycin gel composition suitable for use in the treatment of microcystic LM, as disclosed herein, may comprise isopropyl alcohol in an amount of about 15 wt. %.

In any embodiment, an anhydrous rapamycin gel composition suitable for use in the treatment of microcystic LM, as disclosed herein, may comprise polyethylene glycol as one of the one or more solvents in an amount of about 10 wt. % to about 60 wt. %, about 10 wt. % to about 50 wt. %, about 10 wt. % to about 45 wt. %, about 15 wt. % to about 60 wt. %, about 15 wt. % to about 55 wt. %, about 20 wt. % to about 60 wt. %, about 20 wt. % to about 55 wt. %, about 25 wt. % to about 55 wt. %, about 30 wt. % to about 55 wt. %, or about 40 wt. % to about 60 wt. %. Preferably, an anhydrous rapamycin gel composition suitable for use in the treatment of microcystic LM, as disclosed herein, may comprise polyethylene glycol 400 in an amount of about 54.9 wt. %.

In any embodiment, an anhydrous rapamycin gel composition suitable for use in the treatment of microcystic LM, as disclosed herein, may comprise diisopropyl adipate as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, about 5 wt. % to about 10 wt. %. Preferably, an anhydrous rapamycin gel composition suitable for use in the treatment of microcystic LM, as disclosed herein, may comprise diisopropyl adipate in an amount of about 15 wt. %.

In any embodiment, an anhydrous rapamycin gel composition, suitable for use in the treatment of microcystic LM, as disclosed herein, may comprise glycerol as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, or about 5 wt. % to about 10 wt. %. Preferably, an anhydrous rapamycin gel composition, suitable for use in the treatment of microcystic LM, as disclosed herein, may comprise glycerol in an amount of about 10 wt. %.

In any embodiment, an anhydrous rapamycin gel composition, suitable for use in the treatment of microcystic LM, as disclosed herein, may comprise propylene glycol as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, or about 5 wt. % to about 10 wt. %.

In any embodiment, an anhydrous rapamycin gel composition, suitable for use in the treatment of microcystic LM, as disclosed herein, may comprise benzyl alcohol as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, or about 5 wt. % to about 10 wt. %.

An anhydrous rapamycin gel composition, suitable for use in the treatment of microcystic LM, as disclosed herein, may comprise DMSO as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, or about 5 wt. % to about 10 wt. %. In any embodiment, however, an anhydrous rapamycin gel composition, suitable for use in the treatment of microcystic LM, as disclosed herein, may be substantially free of or free of DMSO.

In any embodiment, an anhydrous rapamycin gel composition, suitable for use in the treatment of microcystic LM, as disclosed herein, may comprise diglycol as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, or about 5 wt. % to about 10 wt. %.

In any embodiment, an anhydrous rapamycin gel composition, suitable for use in the treatment of microcystic LM, as disclosed herein, may comprise propylene glycol monocaprylate as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, or about 5 wt. % to about 10 wt. %.

In any embodiment, an anhydrous rapamycin gel composition, suitable for use in the treatment of microcystic LM, as disclosed herein, may comprise diethylene glycol monoethylether as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, about 5 wt. % to about 10 wt. %.

In any embodiment, an anhydrous rapamycin gel composition, suitable for use in the treatment of microcystic LM, as disclosed herein, may comprise tetrahydrofurfurylalcohol polyethylene glycol ether as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, about 5 wt. % to about 10 wt. %.

In any embodiment, an anhydrous rapamycin gel composition, suitable for use in the treatment of microcystic LM, as disclosed herein, may comprise butylene glycol as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, about 5 wt. % to about 10 wt. %.

In any embodiment, an anhydrous rapamycin gel composition, suitable for use in the treatment of microcystic LM, as disclosed herein, may comprise diethylene glycol as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, about 5 wt. % to about 10 wt. %.

In any embodiment, an anhydrous rapamycin gel composition, suitable for use in the treatment of microcystic LM, as disclosed herein, may comprise triethylene glycol as one of the one or more solvents in an amount of about 5 wt. % to about 55 wt. %, about 5 wt. % to about 50 wt. %, about 5 wt. % to about 45 wt. %, about 5 wt. % to about 40 wt. %, about 5 wt. % to about 35 wt. %, about 5 wt. % to about 30 wt. %, about 5 wt. % to about 25 wt. %, about 5 wt. % to about 20 wt. %, about 5 wt. % to about 15 wt. %, about 5 wt. % to about 10 wt. %.

Preferably, an anhydrous rapamycin gel composition, suitable for use in the treatment of microcystic LM, as disclosed herein, comprises one or more solvents selected from polyethylene glycol, isopropyl alcohol, diisopropyl adipate, glycerol, and combinations thereof. Preferably, if polyethylene glycol is included, polyethylene glycol is present in an amount of about 40 wt. % to about 60 wt. %. Preferably, if isopropyl alcohol is included, isopropyl alcohol is present in an amount of about 10 wt. % to about 20 wt. %. Preferably, if diisopropyl adipate is included, diisopropyl adipate is present in an amount of about 10 wt. % to about 20 wt. %. Preferably, if glycerol is included, glycerol is present in an amount of about 5 wt. % to about 20 wt. %.

### Gelling Agent

In any embodiment, an anhydrous rapamycin gel composition, suitable for use in the treatment of microcystic LM, as disclosed herein, may comprise a gelling agent in an amount of about 0.1 wt. % to about 5 wt. %, about 0.1 wt. % to about 4.5 wt. %, about 0.1 wt. % to about 4 wt. %, about 0.1 wt. % to about 3.5 wt. %, about 0.1 wt. % to about 3 wt. %, about 0.1 wt. % to about 2.5 wt. %, about 0.1 wt. % to about 2 wt. %, about 0.1 wt. % to about 1.5 wt. %. Preferably, an anhydrous rapamycin gel composition, suitable for use in the treatment of microcystic LM, as disclosed herein, comprises about 0.1 wt. % to about 1 wt. % of gelling agent.

Examples of suitable gelling agents include, but are not limited to, hydroxypropyl cellulose, carbomer 981, carbomer 934P, glyceryl tris-12-hydroxy stearate, hydroxy stearin, propylene carbonate, polyvinyl pyrrolidone, and combinations thereof. In any embodiment, an anhydrous rapamycin gel composition, suitable for use in the treatment of microcystic LM, as disclosed herein, may comprise a gelling agent, such as poloxamers and carbomers. Non-limiting examples of poloxamers are poloxamer P-188, poloxamer P-138, poloxamer P-237, poloxamer P-288, poloxamer P-124, poloxamer P-338, and poloxamer P-407. Other block copolymers, such as poly(ethylene glycol/D,L-lactide-co-glyceride) poly(.quadrature.-caprolactum), and hydroxypropyl cellulose (KLUCEL™), glyceryl tris-12-hydroxy stearate, hydroxy stearin, propylene carbonate, polyvinyl pyrrolidone can also be used as gelling agents. Non-limiting examples of carbomers that may be used are carbomer 981, carbomer 934, carbomer 934P, carbomer 940, carbomer 941, carbomer 1342, polycarbophil, and calcium polycarbophil. In a preferred embodiment, the gelling agent is selected from hydroxypropyl cellulose, carbomer 981, carbomer 934P, glyceryl tris-12-hydroxy stearate, hydroxy stearin, propylene carbonate, polyvinyl pyrrolidone, and combinations thereof. Preferably, an anhydrous rapamycin gel composition, suitable for use in the treatment of microcystic LM, as disclosed herein, comprises about 0.1 wt. % to about 1 wt. % hydroxypropyl cellulose.

### Antioxidants

In any embodiment, an anhydrous rapamycin gel composition, suitable for use in the treatment of microcystic LM, as disclosed herein, may comprise an antioxidant in an amount of about 0.001 wt. % to about 1 wt. %, about 0.001 wt. % to about 0.5 wt. %, about 0.001 wt. % to about 0.1 wt. %, about 0.001 wt. % to about 0.05 wt. %, or about 0.001 wt. % to about 0.01 wt. %. In any embodiment, an anhydrous rapamycin gel composition, suitable for use in the treatment of microcystic LM, as disclosed herein, may comprise an antioxidant, such as ascorbic acid, vitamin E and its derivatives, α-tocopherol, Ψ-tocopherol, Δ-tocopherol, ascorbyl palmitate, propyl gallate (PG), octyl gallate, dodecyl gallate, butylated hydroxy anisole (BHA), butylated hydroxy toluene (BHT), D-α-tocopheryl polyethylene glycol 1000 succinate, or combinations thereof. Preferably, an anhydrous rapamycin gel composition, suitable for use in the treatment of microcystic LM, as disclosed herein, may comprise a mixture of antioxidants comprising propyl gallate (PG), ascorbyl palmitate, and α-tocopherol. Preferably, the total amount of antioxidant in an anhydrous rapamycin gel composition, suitable for use in the treatment of microcystic LM, as disclosed herein, is about 0.05 wt. % to about 0.1 wt. %. Preferably, an anhydrous rapamycin gel composition, suitable for use in the treatment of microcystic LM, as disclosed herein, may comprise propyl gallate (PG) in an amount of about 0.01 wt. % to about 0.1 wt. %. Preferably, an anhydrous rapamycin gel composition, suitable for use in the treatment of microcystic LM, as disclosed herein, may comprise ascorbyl palmitate about 0.01 wt. % to about 0.05 wt. %. Preferably, an anhydrous rapamycin gel composition, suitable for use in the treatment of microcystic LM, as disclosed herein, may comprise one or more of α-tocopherol, ascorbyl palmitate, and propyl gallate in an amount of about 0.01 wt. % to about 0.1 wt. %.

### Formulations

In one example, an anhydrous rapamycin gel composition suitable for use in the treatment of microcystic LM, as disclosed herein, may comprise about 2 wt. % to about 6 wt. % rapamycin, about 5 wt. % to about 30 wt. % isopropyl alcohol, about 40 wt. % to about 60 wt. % polyethylene glycol 400, about 5 wt. % to about 30 wt. % diisopropyl adipate, about 5 wt. % to about 30 wt. % glycerol, about 0.5 wt. % to about 1.5 wt. % of hydroxypropyl cellulose, one or more antioxidants in a combined amount of 0.05 wt. % to about 0.1 wt. %, and a buffer. For example, the buffer may be citric acid present in an amount of less than about 0.1 wt. %.

In one example, an anhydrous rapamycin gel composition suitable for use in the treatment of microcystic LM, as disclosed herein, may comprise rapamycin present at about 3.9 wt. %, isopropyl alcohol present at about 15 wt. %, polyethylene glycol 400 present at about 54.9 wt. %, diisopropyl adipate present at about 15 wt. %, glycerol present at about 10 wt. %, hydroxypropyl cellulose present at about 1 wt. %, propyl gallate present at about 0.05 wt. %, ascorbyl palmitate present at about 0.02 wt. %, alpha-tocopherol present at about 0.002 wt. %, and a buffer. For example, the buffer may be citric acid present in an amount of less than about 0.1 wt. %.

Preferably, an anhydrous rapamycin gel composition suitable for use in the treatment of microcystic LM, as disclosed herein, does not contain water. In any embodiment, an anhydrous rapamycin gel composition, suitable for use in the treatment of microcystic LM, as disclosed herein, may contain less than 1 wt. % of water, such as less than 0.5 wt. % of water, less than 0.1 wt. % of water, or substantially no detectable water.

Preferably, an anhydrous rapamycin gel composition, suitable for use in the treatment of microcystic LM, as disclosed herein, does not comprise any penetration enhancer.

The method of dispensing an anhydrous rapamycin gel composition suitable for use in the treatment of microcystic LM, as disclosed herein, is not particularly limited. For example, a hand pump may be used to dispense an anhydrous rapamycin composition. For example, the hand pump may be configured to dispense the required dose of rapamycin within a tolerance specified by a corresponding label approved by a government regulatory agency. The hand pump may deliver 0.5-10 mL of the composition per pump action, such as 1, 2, 3, 4, or 5 mL of the composition per pump action. In any embodiment, an anhydrous rapamycin gel composition may be packaged along with a pharmaceutically acceptable hand pump.

In another example, a liquid medication dispenser may be used to dispense an anhydrous rapamycin gel composition suitable for use in the treatment of microcystic LM, as disclosed herein. liquid medication dispensing systems typically have two methods of dispensing the product, either by using a collapsible bag type design or by using a follower piston-type design. With the collapsible bag type design, a collapsing bag is attached to the dispensing pump, which progressively collapses as the contents are removed. In the piston-type design, a rigid container, usually cylindrical or oval in form, has a follower piston that progressively reduces the container volume as product is drawn out by the dispensing pump. In another example, an anhydrous rapamycin gel composition may be dispensed using an airless pouch, pump-actuating, container system.

In some embodiments, administration of the anhydrous rapamycin gel composition described herein achieves a Cₘₐₓ of about 120-990 micromolar, about 120-900 micromolar, about 600-900 micromolar in the epidermis. In some embodiments, administration of the anhydrous rapamycin gel composition described herein achieves a Cₘₐₓ of about 50-200 micromolar in the dermis.

An anhydrous rapamycin gel composition suitable for use in the treatment of microcystic LM, as disclosed herein, is generally that of a thick liquid or gel but can reach a paste like consistency. Generally, the viscosity may be a minimum of about 5,000 cP, 10,000 cP, or 15,000 cP, preferably about 20,000 cP to a maximum of about 12,000,000 cP, 2,000,000 cP, or even about 600,000 cP.

Advantageously, the rapamycin in an anhydrous rapamycin gel composition, as disclosed herein, is stable for extended periods of time. For example, rapamycin may be stable in an anhydrous rapamycin gel composition, as disclosed herein, at a temperature of about 4° C. to about 50° C., 4° C. to about 45° C., 4° C. to about 40° C., 4° C. to about 35° C., or 4° C. to about 30° C. for a period of 12-36 months.

Definitions and Terminology: The following definitions and description applies to any and all embodiments and aspects of the invention as described herein unless explicitly indicated otherwise.

As used herein, “effective amount” or “therapeutically effective amount” of rapamycin, refers to a sufficient amount of inhibitor to perform an intended task and achieve an intended result. For example, a therapeutically effective amount of rapamycin may be an amount which is sufficient to treat a particular target indication, e.g., microcystic LM, or other condition for which an mTOR inhibitor can be used. It is understood that various biological factors may affect the ability of a particular agent to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. The terms “subject,” “individual” or “patient” are used interchangeably and as used herein are intended to include human and non-human animals. Non-human animals include all vertebrates, for example, mammals and non-mammals, such as non-human primates, sheep, dogs, rats, cats, cows, horses, chickens, amphibians, and reptiles. Examples of mammals include non-human primates, sheep, dogs, cats, cows, and horses. In some examples, the subject is a human or humans. The methods are suitable for treating humans having a viral infection or disease. The subject may be symptomatic or asymptomatic with respect to the viral infection.

The formulations described herein comprise components or ingredients in amounts reported as wt. %. Unless specified otherwise, wt. % indicates a weight percent, or the weight of the particular ingredient being described divided by the weight of the total formulation, the dividend being multiplied by 100 to produce a percent. In all formulations herein, the formulation with all ingredients therein is considered to be 100%.

As used herein, the term “about” is used to provide flexibility to a numerical range endpoint by providing that a given value may be “a little above” or “a little below” the endpoint. As used herein, the term “about” when immediately preceding a numerical value means a range of plus or minus 10% of that value unless the context of the disclosure indicates otherwise or is inconsistent with such an interpretation. For example, “about 50” means 45 to 55 and “about 25,000” means 22,500 to 27,500.

As used in this disclosure, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

“Comprising” is used herein to mean “including, but not limited to.” While various compositions, methods, and devices are described in terms of “comprising” various components or steps, in any embodiment, the composition or method can also “consist essentially of” or “consist of” the described components or steps, but such embodiments are not explicitly listed and included for the sake of brevity, clarity, and efficiency. In any case, “comprising of” terminology should be interpreted as defining and including essentially closed-member groups as well as fully closed-member groups. Other terms that are used herein to indicate “including, but not limited to” are “including,” “having,” “have,” “contain,” and the like. Similarly, the term “having” should be interpreted as “having at least,” the term “includes” should be interpreted as “includes, but is not limited to”.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, the term “substantially free of” as it refers to the presence or lack of a particular composition or ingredient or component in a given formulation refers to the complete or near complete absence of the ingredient from the formulation such that the ingredient, if present, forms only a minor component or impurity of the formulation. When the phrase “substantially free of” is used, separate from the solvent or solvate of rapamycin, it refers to an amount of the component present which should not solubilize an amount of rapamycin so as to negatively impact the therapeutic effect of the formulation.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C,” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together). In instances where a convention analogous to “at least one of A, B, and C” is used, such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

This disclosure is not limited to the particular systems, devices and methods described, as these may vary. The terminology used in the description is for the purpose of describing the particular versions or embodiments only and is not intended to limit the scope. Variations of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1: A Multicenter, Phase 2, Open-Label Study Evaluating the Safety and Efficacy of Rapamycin 3.9% Topical Gel

Without wishing to be bound by theory, the specific pathophysiology of LM, including microcystic LM, is not well understood, which is why management has historically been empirical. Recently, however, literature has suggested that the occurrence and development of ˜90% of all LM is a result of somatic activating mutations in phosphatidylinositol-3-kinase (PI3KCA), specifically in the segment encoding the p110a catalytic subunit. These mutations result in abnormal and increased activation of the PI3KCA/AKT/mTOR signaling pathway, which subsequently causes lymphatic hyperplasia during lymphangiogenesis. Mechanistically hyperactivation of the PI3K pathway results in endothelial cell proliferation and migration, defective mural cell coverage, and aberrant lymphatic vascular network formation. This hyperplasia ultimately results in the anatomic malformations in lymphatic channels seen in this disease and results in disease presentation at birth.

These overgrown lymphatic vessels associated with microcystic LM connect to the epidermis in the form of vesicles, papules and plaques which leak at the surface (lymphorrhea). The healing skin often leads to hyperkeratosis. These lesions can bleed, which is usually the result of a hyperkeratotic region becoming traumatized, resulting in bright red blood and is distinct from lymphorrhea. Over time, since microcystic LMs are caused by a genetic mutation, the lesions proliferate over time, and with recurrent leaking and healing tend to become more papular and keratotic.

Sirolimus, an mTOR inhibitor, is a macrocyclic lactone (macrolide), produced by the bacterium Streptomyces hygroscopicus. As previously mentioned, the somatic activating mutations in PI3KCA and associated signaling of the downstream pathways are thought to underlie the etiology of all LMs, resulting in endothelial cell proliferation and subsequent formation of aberrant lymphatic vasculature. Sirolimus inhibits mTOR, which is a downstream element of this pathway. In doing so, sirolimus is thought to diminish PI3KCA/AKT/mTOR overactivation, thereby reducing endothelial cell proliferation and subsequently the formation of malformed lymphatic vessels. Additionally, through other mechanisms, sirolimus is thought to reduce lymph fluid formation in the affected tissue, helping to minimize clinical symptoms associated with the LMs.

Despite the serious and potentially lethal consequences of microcystic LM, there are no FDA-approved medications for this disease. In fact, currently available treatment options (surgery, sclerotherapy, laser, cryotherapy) are invasive, can induce further scarring/inflammation, and are not completely efficacious, resulting in high recurrence rates.

Clinical studies have evaluated the safety and efficacy of oral sirolimus in patients with LMs and there are reports of case studies of unapproved compounded topical administration of sirolimus (0.1% to 1%) for patients with microcystic LM. Systematic reviews of the efficacy and safety of sirolimus for the treatment of LM lesions have demonstrated that sirolimus can significantly improve prognoses, with a pooled clinical effectiveness (partial, usually defined as improvement in one of three clinical categories, or complete response usually defined as complete resolution of disease) of 94.9% among patients with LM reported in one review and an effectiveness of 84.5% reported in another. In the review of published studies in patients with LMs, >90% of patients studied were <18 years of age. Thus, the current literature overall provides efficacy information on sirolimus therapy for LMs in both pediatric and adult patients.

However, oral sirolimus is associated with a severe adverse event profile and requires frequent patient monitoring especially for pediatric and adolescent patients, which together limits its use. Side effects of sirolimus are explained by the cellular pathways affected and the product label for both approved indications: organ rejection prophylaxis for patients (>13 years old) receiving renal transplants and lymphangioleiomyomatosis.

The most common (≥30%) adverse reactions observed with RAPAMUNE® in clinical studies for organ rejection prophylaxis in recipients of renal transplantation include: peripheral edema, hypertriglyceridemia, hypertension, hypercholesterolemia, creatinine increased, constipation, abdominal pain, diarrhea, headache, fever, urinary tract infection, anemia, nausea, arthralgia, pain, and thrombocytopenia. Similarly, the most common (≥20%) adverse reactions observed with RAPAMUNE® in clinical studies for the treatment of lymphangioleiomyomatosis were: stomatitis, diarrhea, abdominal pain, nausea, nasopharyngitis, acne, chest pain, peripheral edema, upper respiratory tract infection, headache, dizziness, myalgia, and hypercholesterolemia. Other common side effects reported across both groups of patients included: acne, stomatitis, nasopharyngitis, upper respiratory tract infection and dizziness. A more complete description of the adverse event profile of RAPAMUNE® can be found in the product labeling.

Because there are no FDA approved therapies in LMs and there are clinical studies demonstrating efficacy with oral sirolimus, it is often used in clinical practice and recommended as first line therapy for complicated LMs; however there remains a significant unmet need for a targeted therapy that limits systemic absorption and reduces adverse events compared to oral sirolimus. The participant population for this study was selected with the goal of targeted delivery of sirolimus to the cutaneous lymphatic malformation with limited systemic exposure.

Inclusion criteria for the study, at visit 1, include (1) at least 13 years of age at the screening visit; (2) if less than 18 years of age, the participant and legal guardian must provide written informed consent/assent prior to any study procedures; (3) the participant must have a clinically confirmed microcystic lymphatic malformation (LM); (4) the microcystic LM to be treated (lesion) must have a defined total area of greater than about 25 cm² and less than about 200 cm² (the lesion within the total area does not need to be contiguous); (5) the subject is willing to have blood collected for safety and PK testing; (6) the investigator must identify the presences of vesicles, papules, or plaques consistent with a microcystic LM; (7) the participant reports either bleeding or leaking ≥5 days over the past 14 days; (8) the participant is willing to abstain from application of other topical medications (prescription or over the counter) for the duration of the trial. Moisturizers and emollients are allowed except for the area of the lesion; (9) the participant is willing to forego medical interventional treatment (i.e., pharmacological or interventional such as sclerotherapy) of their microcystic LM with anything other than the study IP except when the Investigator believes that delay of treatment potentially might compromise the health of the subject; (10) at least 4 weeks have elapsed since the participant has had any major surgery; (11) at least 14 days have elapsed since the participant has completed therapy with a growth factor (GF) that supports platelet, red or white cell number or function; (12) participants must not have received any investigational drug or biologic within 4 weeks or 5 half-lives, whichever is longer, prior to starting treatment with and during treatment with anhydrous rapamycin gel (with the exception of drugs, biologics or vaccines for COVID-19 authorized under Emergency Use Authorization.); (13) there has been ≥6 months from involved field radiation (XRT) administered to the microcystic LM that will be studied. During the Screening period: (14) in the Investigator's opinion, the participant's overall health status, including adequate organ function, LDL and cholesterol levels, should be appropriate to permit participation in this study; (15) the participant has adequate renal function with an eGFR greater than 90 mL/min/1.73 m² or CrCl greater than 90 mL/min.

Individuals will be excluded from the study if, at visit 1: (1) The participant has used sirolimus in the past 6 weeks; (2) The participant has previously participated in a clinical trial evaluating an investigational product for treatment of Vascular Anomalies, including microcystic LM in the last 3 months: (3) the participant has used a topical, oral, or interventional treatment that might interfere with the evaluation of the study IP. Among these are use of the following: (a) myelosuppressive chemotherapy within 4 weeks; (b) photodynamic therapy (PDT) or other laser surgeries to target lesions within 3 months; (c) chronic treatment with systemic steroids or another immunosuppressive agent, or (d) the participant has a medically significant infection requiring antibiotic treatment in the past 2 months; (4) the participant requires medications and OTC supplements that inhibit/induce CYP3A4 activity to control concurrent medical conditions; (5) the participant's lesion to be treated is mainly in any wet mucosa or within the orbital rim. External genital presentation is permitted; (6) the participant has known hypersensitivity to any of the ingredients in the study medication formulation; (7) the participant has a known history of HIV seropositivity or known immunodeficiency; (8) the participant has complicated vascular anomalies with severe systemic symptoms that require systemic therapy; (9) the participant has concurrent severe and/or uncontrolled medical disease which could compromise compliance with safety monitoring requirements for sirolimus; (10) the participant has been previously treated for invasive cancer within the past 5 years unless the Investigator concludes history of cancer is not confounding to safety; (11) the participant is a woman who is pregnant, breastfeeding or planning to become pregnant during the study including the follow-up period; (12) Participants of childbearing potential who are unwilling or unable to comply with contraception measures; (13) the participant has any condition or situation which, in the Investigator's opinion, may put the subject at significant risk, could confound the study results, or could interfere significantly with the subject's participation in the study; (14) Participants deemed by the investigator as unwilling or unable to remain compliant with all tests and procedures, adherence to the study drug administration regimen and other protocol-required activities; and (15) participant has had either COVID-19 or the COVID-19 Vaccine within the last 6 weeks.

Open-Label Treatment Period Inclusion Criteria: 1) Completion of the Screening Period; 2) Completion of the Baseline Period: a) Participant averaged leaking or bleeding for ≥5 out of the last 14 days prior to Day 1, or b) Participant had leaking or bleeding for any 9 out of 14 days; 3) Maintained >70% compliance with completion of the PDD during the Baseline period. Open-Label Treatment Period Exclusion Criteria: 1) Participants deemed by the investigator as unwilling or unable to remain compliant with all tests and procedures, including adherence to the study drug administration regimen and other protocol-required activities.

The anhydrous rapamycin gel has formulation as described in Table 1 below.

TABLE 1

| Component | Concentration (wt. %) |
|---|---|
| isopropyl alcohol | 15.0 |
| PEG400 | 54.9 |
| diisopropyladipate | 15.0 |
| Glycerol | 10.0 |
| propyl gallate | 0.05 |
| ascorbyl palmitate | 0.2 |
| α-tocopherol | 0.002 |
| citric acid | <0.1 |
| hydroxypropylcellulose | 0.75 |
| rapamycin | 3.9 |
| Total | 100 |

All subjects complete 12 weeks of therapy, which entails topically administration of the anhydrous rapamycin gel in the evening, at least one hour before bed. The anhydrous rapamycin gel is applied once daily to the lesion area only, not exceeding 200 cm². Specific instructions are available in the Participant Instruction for Use manual. If the area is less than 200 cm², the gel is applied to cover the target area(s) and any remaining gel is discarded.

Safety is assessed through adverse event (AE) evaluations. During the study, subjects are assessed for the occurrence of new and ongoing AEs. Descriptions of AEs include the dates of onset and resolution (if resolved), maximum severity, and seriousness, action taken regarding the study drug, corrective treatment, outcome, and the Investigator's assessment of causality. AEs present at any visit are followed to resolution (return to normal or to the baseline state) or until clinically stable as determined by the Investigator.

### Efficacy Assessments Include:

Clinician Global Impression of Severity (CGI-S)
Clinician Global Impression of Change (CGI-C)
Patient Global Impression of Severity (PGI-S)
Patient Global Impression of Change (PGI-C)
Patient Global Impression of Severity (PGI-SL) for leaking
Patient Global Impression of Severity (PGI-SB) for bleeding
Number of days in which lesion leaking is reported
Number of days in which lesion bleeding is reported
Number of days in which lesion leaking or bleeding is reported
Change in the lesion impact on function
Severity of lesion pain, crusting or redness based on the LM Symptom Severity Scale
Dermatology Quality of Life Index (DQLI) (participants≥16 years) or the Children's Dermatology Quality of Life Index (participants≤15 years)
Thickness/height or size of the lesion
Overall participant satisfaction

### Analysis Populations:

Safety/Intent to Treat (ITT) population: All subjects who received at least one application of study intervention. Per Protocol Population: All subjects who completed treatment without major protocol violations affecting the efficacy endpoints.

### Efficacy Endpoint/s are as Follows:

Change in CGI-S from baseline to Day 84. CGI-S will be separately measured over the following domains:
   Height of lesion (includes vesicles, papules, or plaques)
   Appearance of lymphorrhea
   Appearance of bleeding
   Appearance of crusting/hyperkeratosis
   Appearance of erythema
   Overall severity
CGI-C from baseline to Day 84.
Change in the frequency of leaking from baseline to Day 84
Change in the frequency of bleeding from baseline to Day 84
Change in the PGI-SL for leaking from baseline to Day 84
Change in PGI-SB for bleeding from baseline to Day 84
Change in the frequency of leaking or bleeding from baseline to Day 84
Change in the participant reported symptoms from baseline to Day 84. The following symptoms will be measured:
   Pain
   Crusting
Change in the overall PGI-S from baseline to Day 84
PGI-C.
PGI-CL
PGI-CB
Change in the lesion impact on function from baseline to Day 84
Change in the 10-question dermatology quality of life index (participants≥16 years) or the children dermatology quality of life index (participants≤15 years)
Change in height or size of the lesion as measured by photography
Overall participant satisfaction as measured by TSQM

### Example 2: Assessments

### Clinician Global Impression of Severity (CGI-S)

The CGI-S provides an overall clinician-determined summary measure of disease activity. Clinicians will complete the CGI-S on a 5-point Likert scale at each visit. The clinician will take into account data available at the time of the assessment.

Below is the CGI-S question and scale that will be presented to the clinician. Each domain questions should be answered independently:
For each domain, please choose the response that best describes your assessment of the treatment area disease severity, based upon the totality of information available to you.

| Domain | Clear | Mild | Moderate | Severe | Very Severe |
|---|---|---|---|---|---|
| Overall severity | 1 | 2 | 3 | 4 | 5 |

| Domain | Clear | Mild | Moderate | Severe | Very Severe |
|---|---|---|---|---|---|
| Height of lesion | 1 | 2 | 3 | 4 | 5 |

| Domain | Clear | Mild | Moderate | Severe | Very Severe |
|---|---|---|---|---|---|
| Visual observation of | 1 | 2 | 3 | 4 | 5 |
| leaking or oozing | | | | | |

| Domain | Clear | Mild | Moderate | Severe | Very Severe |
|---|---|---|---|---|---|
| Visual obversion of | 1 | 2 | 3 | 4 | 5 |
| bleeding | | | | | |

| Domain | Clear | Mild | Moderate | Severe | Very Severe |
|---|---|---|---|---|---|
| Visual observation of | 1 | 2 | 3 | 4 | 5 |
| erythema | | | | | |

| Domain | Clear | Mild | Moderate | Severe | Very Severe |
|---|---|---|---|---|---|
| Visual observation of | 1 | 2 | 3 | 4 | 5 |
| crusting/hyperkeratosis | | | | | |

### Clinician Global Impression of Change (CGI-C)

The Clinician Global Impression of Change (CGI-C), a 7-point Likert scale, will be used.

| −3 | −2 | −1 | 0 | +1 | +2 | +3 |
|---|---|---|---|---|---|---|
| Very Much | Much | Minimally | No Change | Minimal | Much | Very Much |
| Worse | Worse | Worse | | Improvement | Improvement | Improved |

CGI-C will be completed by the investigator, on paper, at various timepoints. While the 7-point Likert scale will remain the same, the question being asked during each time period will vary and refer to the same baseline period.

### Patient Global Impression of Change (PGI-C)

The Patient Global Impression of Change (PGI-C), a 7-point Likert scale, will be used.

| −3 | −2 | −1 | 0 | +1 | +2 | +3 |
|---|---|---|---|---|---|---|
| Very Much | Much | Minimally | No Change | Minimal | Much | Very Much |
| Worse | Worse | Worse | | Improvement | Improvement | Improved |

PGI-C will be completed by the investigator, on paper, at various timepoints. While the 7-point Likert scale will remain the same, the question being asked during each time period will vary and refer to the same baseline period.

### Patient Global Impression of Severity (PGI-S)

The participants' overall impression of severity of their lesion (Patient Global Impression of Severity) will be self-reported and captured on a 5-point Likert scale at each study visit. Below is the question and scale that will be presented to the participant:
Please choose the response below that best describes the severity of your lesion over the past week:
   None
   Mild
   Moderate
   Severe
   Very severe

In addition to participants' overall impression of severity, participants will also self-report their impression of the severity of leaking (PGI-SL) and the severity of bleeding (PGI-SB), weekly in the PDD
Please choose the response below that best describes the severity of lesion leaking you experienced over the past week:
   None
   Mild
   Moderate
   Severe
   Very severe
Please choose the response below that best describes the severity of lesion bleeding you experienced over the past week:
   None
   Mild
   Moderate
   Severe
   Very severe

### Microcystic LM Symptom Severity Scale

In addition to the overall impression of severity, participants will be asked, in the PDD, to rate the severity of pain (daily) and to rate the severity of crusting and redness symptoms (weekly) on an 11-point LM Symptom Severity Scale:
On a scale of 0-10, where 0 is clear/no symptoms and 10 is very severe, please rate the average severity of the following symptoms related to your lesion over the past week.

| | | | | | | | | | | | Pain as bad |
|---|---|---|---|---|---|---|---|---|---|---|---|
| | No Pain | | | | | | | | | | as you can imagine |
| Symptom | 0 | 1 | 2 | 3 | 4 | 5 | 6 | 7 | 8 | 9 | 10 |
| Worst Pain (daily) | | | | | | | | | | | |

| | Clear | | | | | | | | | | Very Severe |
|---|---|---|---|---|---|---|---|---|---|---|---|
| Symptom | 0 | 1 | 2 | 3 | 4 | 5 | 6 | 7 | 8 | 9 | 10 |
| Crusting (weekly) | | | | | | | | | | | |
| Redness (weekly) | | | | | | | | | | | |

### Assessment of Lesion Leakage

Participants will be asked daily, in the PDD, whether they experienced leakage from their LM lesion(s) identified for the study.
Did you experience leakage from the LM lesion(s) identified for the study within the past 24 hours?
   Yes
   No

### Assessment of Lesion Bleeding

Participants will be asked daily, in the PDD, whether they experienced bleeding from their LM lesion(s) identified for the study.
Did you experience bleeding from the LM lesion(s) identified for the study within the past 24 hours?
   Yes
   No

### Assessment of Lesion on Function

Participants will be asked daily, in the PDD, whether the bleeding or leakage from their LM lesion(s) impacted their daily function.
Did any leaking from the LM lesion(s) identified for the study interfere with your ability to conduct day to day activities within the past 24 hours?
   Very much
   Quite a bit
   Somewhat
   A little bit
   Not at all
Did any bleeding from the LM lesion(s) identified for the study interfere with your ability to conduct day to day activities within the past 24 hours?
   Very much
   Quite a bit
   Somewhat
   A little bit
   Not at all

### Change in the Lesion Size

Lesion size and height will be measured using 3D digital photography according to the SoA and the photography manual.

### Dermatology Quality of Life and Children's Dermatology Quality of Life (DQLI)

The aim of this questionnaire is to measure how much your skin problem has affected
your life OVER THE LAST WEEK. Please check one box for each question.

| | | | | |
|---|---|---|---|---|
| 1. | Over the last week, how itchy, sore, | Very much | □ | |
| | painful or stinging has your skin been? | A lot | □ | |
| | | A little | □ | |
| | | Not at all | □ | |
| 2. | Over the last week, how embarrassed or self- | Very much | □ | |
| | conscious have you been because of your skin? | A lot | □ | |
| | | A little | □ | |
| | | Not at all | □ | |
| 3. | Over the last week, how much has your | Very much | □ | Not relevant □ |
| | skin interfered with you going shopping or | A lot | □ | |
| | looking after your home or yard? | A little | □ | |
| | | Not at all | □ | |
| 4. | Over the last week, how much has your skin | Very much | □ | Not relevant □ |
| | influenced the clothes you wear? | A lot | □ | |
| | | A little | □ | |
| | | Not at all | □ | |
| 5. | Over the last week, how much has your skin | Very much | □ | Not relevant □ |
| | affected any social or leisure activities? | A lot | □ | |
| | | A little | □ | |
| | | Not at all | □ | |
| 6. | Over the last week, how much has your skin | Very much | □ | Not relevant □ |
| | made it difficult for you to do any sport? | A lot | □ | |
| | | A little | □ | |
| | | Not at all | □ | |
| 7. | Over the last week, has your skin prevented | yes | □ | Not relevant □ |
| | you from working or studying? | no | □ | |
| | If “No”, over the last week how much has your | A lot | □ | |
| | skin been a problem at work or studying? | A little | □ | |
| | | Not at all | □ | |
| 8. | Over the last week, how much has your skin | Very much | □ | Not relevant □ |
| | created problems with your partner or any | A lot | □ | |
| | of your close friends or relatives? | A little | □ | |
| | | Not at all | □ | |
| 9. | Over the last week, how much has your skin | Very much | □ | Not relevant □ |
| | caused any sexual difficulties? | A lot | □ | |
| | | A little | □ | |
| | | Not at all | □ | |
| 10. | Over the last week, how much of a problem has | Very much | □ | Not relevant □ |
| | the treatment for your skin been, for example | A lot | □ | |
| | by making your home messy, or by taking up time? | A little | □ | |
| | | Not at all | □ | |

The aim of this questionnaire is to measure how much your skin problem has
affected you OVER THE LAST WEEK. Please tick ✓ one box for each question.

| 1. | Over the last week, how itchy, “scratchy”, | | | Very much | □ |
|---|---|---|---|---|---|
| | sore or painful has your skin been? | | | Quite a lot | □ |
| | | | | Only a little | □ |
| | | | | Not at all | □ |
| 2. | Over the last week, how embarrassed | | | Very much | □ |
| | or self conscious, upset or sad have you | | | Quite a lot | □ |
| | been because of your skin? | | | Only a little | □ |
| | | | | Not at all | □ |
| 3. | Over the last week, how much has your | | | Very much | □ |
| | skin affected your friendships? | | | Quite a lot | □ |
| | | | | Only a little | □ |
| | | | | Not at all | □ |
| 4. | Over the last week, how much have you changed | | | Very much | □ |
| | or worn different or special clothes/shoes | | | Quite a lot | □ |
| | because of your skin? | | | Only a little | □ |
| | | | | Not at all | □ |
| 5. | Over the last week, how much has your | | | Very much | □ |
| | skin trouble affected going out, playing, | | | Quite a lot | □ |
| | or doing hobbies? | | | Only a little | □ |
| | | | | Not at all | □ |
| 6. | Over the last week, how much have you | | | Very much | □ |
| | avoided swimming or other sports because | | | Quite a lot | □ |
| | of your skin trouble? | | | Only a little | □ |
| | | | | Not at all | □ |
| 7. | Last week, | [symbol] | If school time: Over the | Prevented school | □ |
| | was it | | last week, how much did | Very much | □ |
| | school time? | | your skin problem affect your | Quite a lot | □ |
| | | | school work? | Only a little | □ |
| | OR | | | Not at all | □ |
| | was it | [symbol] | If holiday time: How much | Very much | □ |
| | holiday time? | | over the last week, has your | Quite a lot | □ |
| | | | skin problem interfered with | Only a little | □ |
| | | | your enjoyment of the holiday? | Not at all | □ |
| 8. | Over the last week, how much trouble | | | Very much | □ |
| | have you had because of your skin with | | | Quite a lot | □ |
| | other people calling you names, teasing, | | | Only a little | □ |
| | bullying, asking questions or avoiding you? | | | Not at all | □ |
| 9. | Over the last week, how much has your sleep | | | Very much | □ |
| | been affected by your skin problem? | | | Quite a lot | □ |
| | | | | Only a little | □ |
| | | | | Not at all | □ |
| 10. | Over the last week, how much of a | | | Very much | □ |
| | problem has the treatment for your | | | Quite a lot | □ |
| | skin been? | | | Only a little | □ |
| | | | | Not at all | □ |
| 1. | Over the last week, how itchy, “scratchy”, | | | Very much | □ |
| | sore or painful has your skin been? | | | Quite a lot | □ |
| | | | | Only a little | □ |
| | | | | Not at all | □ |
| 2. | Over the last week, how embarrassed | | | Very much | □ |
| | or self conscious, upset or sad have you | | | Quite a lot | □ |
| | been because of your skin? | | | Only a little | □ |
| | | | | Not at all | □ |
| 3. | Over the last week, how much has your | | | Very much | □ |
| | skin affected your friendships? | | | Quite a lot | □ |
| | | | | Only a little | □ |
| | | | | Not at all | □ |
| 4. | Over the last week, how much have you changed | | | Very much | □ |
| | or worn different or special clothes/shoes | | | Quite a lot | □ |
| | because of your skin? | | | Only a little | □ |
| | | | | Not at all | □ |
| 5. | Over the last week, how much has your | | | Very much | □ |
| | skin trouble affected going out, playing, | | | Quite a lot | □ |
| | or doing hobbies? | | | Only a little | □ |
| | | | | Not at all | □ |
| 6. | Over the last week, how much have you | | | Very much | □ |
| | avoided swimming or other sports because | | | Quite a lot | □ |
| | of your skin trouble? | | | Only a little | □ |
| | | | | Not at all | □ |
| 7. | Last week, | [symbol] | If school time: Over the | Prevented school | □ |
| | was it | | last week, how much did | Very much | □ |
| | school time? | | your skin problem affect your | Quite a lot | □ |
| | | | school work? | Only a little | □ |
| | OR | | | Not at all | □ |
| | was it | [symbol] | If holiday time: How much | Very much | □ |
| | holiday time? | | over the last week, has your | Quite a lot | □ |
| | | | skin problem interfered with | Only a little | □ |
| | | | your enjoyment of the holiday? | Not at all | □ |

| | | | |
|---|---|---|---|
| 8. | Over the last week, how much trouble | Very much | □ |
| | have you had because of your skin with | Quite a lot | □ |
| | other people calling you names, teasing, | Only a little | □ |
| | bullying, asking questions or avoiding you? | Not at all | □ |
| 9. | Over the last week, how much has your sleep | Very much | □ |
| | been affected by your skin problem? | Quite a lot | □ |
| | | Only a little | □ |
| | | Not at all | □ |
| 10. | Over the last week, how much of a | Very much | □ |
| | problem has the treatment for your | Quite a lot | □ |
| | skin been? | Only a little | □ |
| | | Not at all | □ |

### Treatment Satisfaction Questionnaire (TSQM)

The Treatment Satisfaction Questionnaire for Medication (TSQM-9) is a measure of treatment satisfaction that allows comparisons across different medication types and health conditions. The purpose of the TSQM-9 in this study is to assess participant satisfaction with study drug. The questionnaire will be completed by participants at Day 84.

TSQM-9
Abbreviated Treatment Satisfaction Questionnaire for Medication
Instructions: Please take some time to think about your level of satisfaction of
dissatisfaction with the medication you are taking in this clinical trial. We are
interested in your evaluation of the effectiveness, side effects, and convenience
of the medication over the last two to three weeks, or since you last used it. For
each question, please place a single check mark next to the response that most
closely corresponds to your own experiences.
1. How satisfied or dissatisfied are you with the ability of the medication to
prevent or treat your condition?

| | |
|---|---|
| □₁ | Extremely Dissatisfied |
| □₂ | Very Dissatisfied |
| □₃ | Dissatisfied |
| □₄ | Somewhat Satisfied |
| □₅ | Satisfied |
| □₆ | Very Satisfied |
| □₇ | Extremely Satisfied |

2. How satisfied or dissatisfied are you with the way the medication relieves
your symptoms?

| | |
|---|---|
| □₁ | Extremely Dissatisfied |
| □₂ | Very Dissatisfied |
| □₃ | Dissatisfied |
| □₄ | Somewhat Satisfied |
| □₅ | Satisfied |
| □₆ | Very Satisfied |
| □₇ | Extremely Satisfied |

3. How satisfied or dissatisfied are you with the amount of time it takes the
medication to start working?

| | |
|---|---|
| □₁ | Extremely Dissatisfied |
| □₂ | Very Dissatisfied |
| □₃ | Dissatisfied |
| □₄ | Somewhat Satisfied |
| □₅ | Satisfied |
| □₆ | Very Satisfied |
| □₇ | Extremely Satisfied |

4. How easy or difficult is it to use the medication in its current form?

| | |
|---|---|
| □₁ | Extremely Difficult |
| □₂ | Very Difficult |
| □₃ | Difficult |
| □₄ | Somewhat Easy |
| □₅ | Easy |
| □₆ | Very Easy |
| □₇ | Extremely Easy |

5. How easy or difficult is it to plan when you will use the medication each time?

| | |
|---|---|
| □₁ | Extremely Difficult |
| □₂ | Very Difficult |
| □₃ | Difficult |
| □₄ | Somewhat Easy |
| □₅ | Easy |
| □₆ | Very Easy |
| □₇ | Extremely Easy |

6. How convenient or inconvenient is it to take the medication as instructed?

| | |
|---|---|
| □₁ | Extremely Inconvenient |
| □₂ | Very Inconvenient |
| □₃ | Inconvenient |
| □₄ | Somewhat Convenient |
| □₅ | Convenient |
| □₆ | Very Convenient |
| □₇ | Extremely Convenient |

7. Overall, how confident are you that taking this medication is a good thing
for you?

| | |
|---|---|
| □₁ | Not at All Confident |
| □₂ | A Little Confident |
| □₃ | Somewhat Confident |
| □₄ | Very Confident |
| □₅ | Extremely Confident |

8. How certain are you that the good things about your medications outweigh
the bad things?

| | |
|---|---|
| □₁ | Not at All Certain |
| □₂ | A Little Certain |
| □₃ | Somewhat Certain |
| □₄ | Very Certain |
| □₅ | Extremely Certain |

9. Taking all things into account, how satisfied or dissatisfied are you
with this medication?

| | |
|---|---|
| □₁ | Extremely Dissatisfied |
| □₂ | Very Dissatisfied |
| □₃ | Dissatisfied |
| □₄ | Somewhat Satisfied |
| □₅ | Satisfied |
| □₆ | Very Satisfied |
| □₇ | Extremely Satisfied |

### Example 3: Subject 1 Results

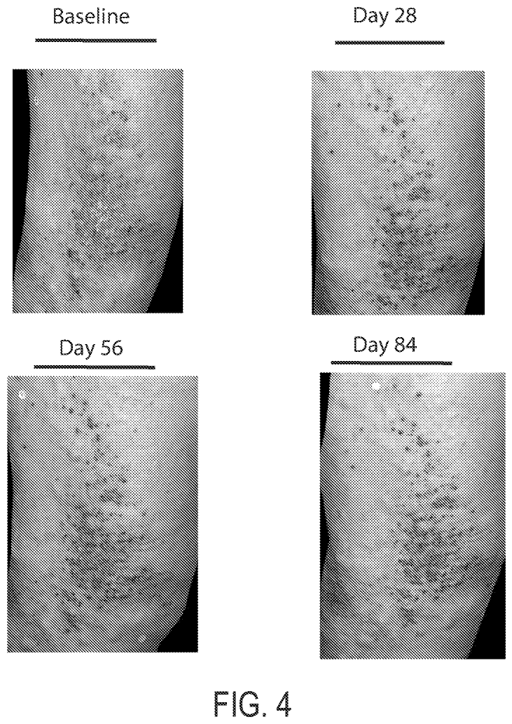

Subject 1 was enrolled in the study with moderate microcystic LM. FIG. 4 shows baseline microcystic LM evaluation and day 28, day 56 and day 84 evaluations following treatment. The clinician assessments at clinical visits (Table 2), patient assessments at clinical visits (Table 3) and patient daily or weekly assessments (Table 4) are shown below.

TABLE 2
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 3 - Moderate | 3 - Moderate | 1 - None | 1 - None | 2 - Mild | 3 - Moderate | N/A |
| Day 28 | 3 - Moderate | 3 - Moderate | 1 - None | 1 - None | 2 - Mild | 2 - Mild | +1 Minimal |
| | | | | | | | Improvement |
| Day 56 | 3 - Moderate | 3 - Moderate | 1 - None | 2 - Mild | 2 - Mild | 2 - Mild | +1 Minimal |
| | | | | | | | Improvement |
| Day 84 | 3 - Moderate | 3 - Moderate | 1 - None | 1 - None | 1 - None | 2 - Mild | +2 Much |
| | | | | | | | Improvement |

TABLE 3
Patient Assessments at Clinical Visits

| | PGI-S | PGI-C | Blood Levels |
|---|---|---|---|
| Baseline | Moderate | N/A | <2 ng/mL |
| Day 28 | Moderate | +1 Minimal | <2 ng/ml |
| Day 56 | Moderate | +1 Minimal | <2 ng/ml |
| Day 84 | Moderate | +1 Minimal | <2 ng/ml |

TABLE 4
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | None | None | N/A | N/A | 0 | 0/14 | 0/14 | 0 |
| Day 28 | None | None | No | No | 0 | 0/14 | 0/14 | 2 |
| | | | Change | Change | | | | |
| Day 56 | None | Moderate | No | No | 5.5 | 0/14 | 0/14 | 4.7 |
| | | | Change | Change | | | | |
| Day 84 | None | None | No | No | 0 | 0/14 | 0/14 | 0.3 |
| | | | Change | Change | | | | |

### Example 4: Subject 2 Results

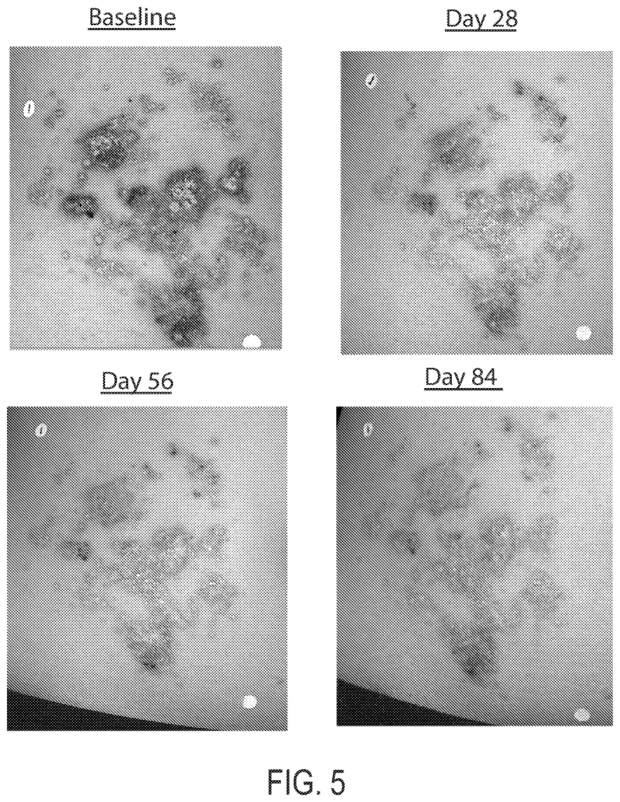

Subject 2 was enrolled in the study with severe microcystic LM. FIG. 5 shows baseline of microcystic LM evaluation and day 28, day 56 and day 84 evaluations following treatment. The clinician assessments at clinical visits (Table 5), patient assessments at clinical visits (Table 6) and patient daily or weekly assessments (Table 7) are shown below.

TABLE 5
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 4 - Severe | 4 - Severe | 2 - Mild | 1 - None | 4 - Severe | 3 - Moderate | N/A |
| Day 28 | 3 - Moderate | 3 - Moderate | 1 - None | 1 - None | 3 - Moderate | 2 - Mild | +1 Minimal |
| | | | | | | | Improvement |
| Day 56 | 2 - Mild | 2 - Mild | 1 - None | 1 - None | 2 - Mild | 2 - Mild | +2 Much |
| | | | | | | | Improvement |
| Day 84 | 2 - Mild | 2 - Mild | 1 - None | 1 - None | 2 - Mild | 1 - None | +3 Very |
| | | | | | | | Much |
| | | | | | | | Improved |

TABLE 6
Patient Assessments at Clinical Visits

| | PGI-S | PGI-C | Blood Levels |
|---|---|---|---|
| Baseline | Moderate | N/A | <2 ng/ml |
| Day 28 | Moderate | +1 minimal improvement | <2 ng/ml |
| Day 56 | Moderate | +2 Much Improvement | <2 ng/ml |
| Day 84 | Moderate | +2 Much Improvement | <2 ng/ml |

TABLE 7
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | None | None | N/A | N/A | 2.5 | 1/14 | 2/14 | 0.14 |
| Day 28 | None | None | No Change | No Change | 3.5 | 0/14 | 1/14 | 2.3 |
| Day 56 | | | | | | 0/8 | 1/8 | 0.6 |
| Day 84 | None | None | Much | Much | 1.0 | 0/12 | 0/12 | 0.0 |
| | | | Improvement | Improvement | | | | |

### Example 5: Subject 3 Results

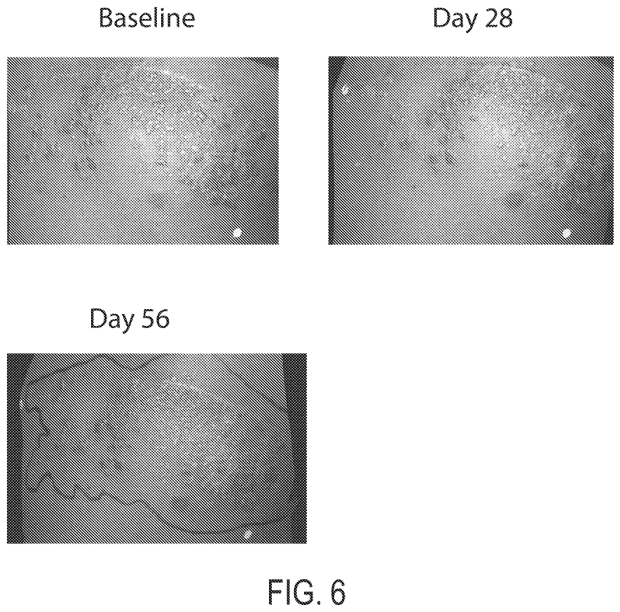

Subject 3 was enrolled in the study with moderate microcystic LM. FIG. 6 shows of microcystic LM baseline evaluation and day 28 and day 56 evaluations following treatment. The clinician assessments at clinical visits (Table 8), patient assessments at clinical visits (Table 9) and patient daily or weekly assessments (Table 10) are shown below.

TABLE 8
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 3 - | 4 - | 1 - | 1 - | 1 - | 3 - | N/A |
| | Moderate | Severe | None | None | None | Moderate | |
| Day 28 | 3 - | 3 - | 1 - | 1 - | 1 - | 1 - | +1 Minimal |
| | Moderate | Moderate | None | None | None | None | Improvement |
| Day 56 | 2 - | 2 - | 1 - | 1 - | 1 - | 1 - | +2 Much |
| | Mild | Mild | None | None | None | None | Improvement |
| Day 84 | | | | | | | |

TABLE 9
Patient Assessments at Clinical Visits

| | PGI-S | PGI-C | Blood Levels |
|---|---|---|---|
| Baseline | Moderate | N/A | <2 ng/ml |
| Day 28 | Mild | +1 Minimal Improvement | <2 ng/ml |
| Day 56 | None | +2 Much Improvement | <2 ng/ml |
| Day 84 | | | |

TABLE 10
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | None | None | N/A | N/A | 0 | 0/14 | 0/14 | 1.6 |
| Day 28 | None | None | No | No | 2.5 | 0/12 | 0/12 | 2.5 |
| | | | Change | Change | | | | |
| Day 56 | None | None | No | No | 0 | 0/11 | 0/11 | 0 |
| | | | Change | Change | | | | |
| Day 84 | | | | | 0 | 0/3 | 0/3 | 0 |

### Example 6: Subject 4 Results

Subject 4 was enrolled in the study with moderate microcystic LM. Baseline of microcystic LM evaluation and day 28, day 56 and day 84 evaluations following treatment were made as per protocol. The clinician assessments at clinical visits (Table 11), patient assessments at clinical visits (Table 12) and patient daily or weekly assessments (Table 13) are shown below.

TABLE 11
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 3 - | 3 - | 1 - | 1 - | 2 - | 1 - | N/A |
| | Moderate | Moderate | None | None | Mild | None | |
| Day 28 | 2 - | 1 - | 1 - | 1 - | 1 - | 1 - | +2 Much |
| | Mild | Flat | None | None | None | None | Improved |
| Day 56 | 2 - | 2 - | 1 - | 1 - | 1 - | 1 - | +2 Much |
| | Mild | Mild | None | None | None | None | Improved |
| Day 84 | 2 - | 3 - | 1 - | 1 - | 1 - | 1 - | +2 Much |
| | Mild | Moderate | None | None | None | None | Improved |

TABLE 12
Patient Assessments at Clinical Visits

| | PGI-S | PGI-C | Blood Levels |
|---|---|---|---|
| Baseline | Moderate | N/A | <2 ng/ml |
| Day 28 | Moderate | +1 Minimal Improvement | <2 ng/ml |
| Day 56 | Mild | +1 Minimal Improvement | <2 ng/ml |
| Day 84 | Mild | +2 Much Improved | <2 ng/ml |

TABLE 13
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | None | None | N/A | N/A | 1 | 1/12 | 1/12 | 6.2 |
| Day 28 | None | None | No Change | No Change | 0 | 0/8 | 0/8 | 6.5 |
| Day 56 | Mild | Mild | Much and | Much and | 2.5 | 0/14 | 0/14 | 6.1 |
| | | | Minimal | Minimal | | | | |
| | | | Improvement | Improvement | | | | |
| Day 84 | Mild | Mild | Minimal | Minimal | 2 | 0/14 | 0/14 | 6.3 |
| | | | Improvement | Improvement | | | | |

### Example 7: Subject 5 Results

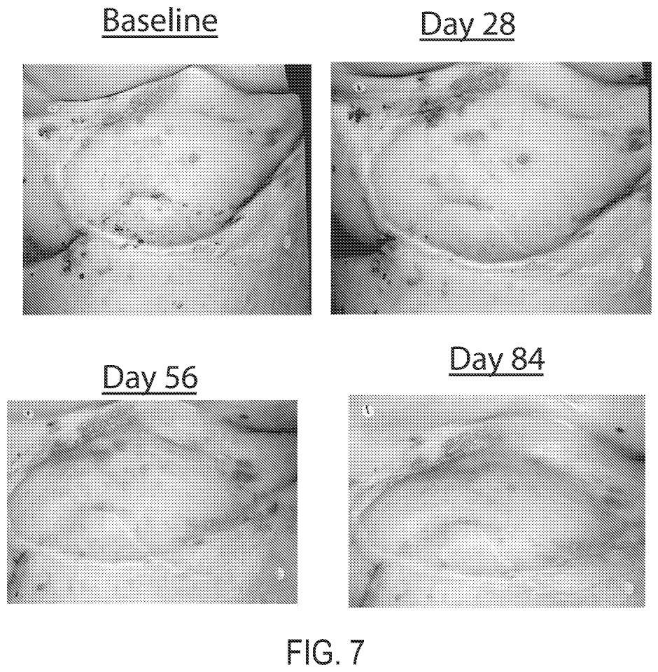

Subject 5 was enrolled in the study with moderate microcystic LM. FIG. 7 shows baseline of microcystic LM evaluation and day 28, day 56 and day 84 evaluations following treatment. The clinician assessments at clinical visits (Table 14), patient assessments at clinical visits (Table 15) and patient daily or weekly assessments (Table 16) are shown below.

TABLE 14
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 3 - | 4 - | 3 - | 2 - | 2 - | 1 - | N/A |
| | Moderate | Severe | Moderate | Mild | Mild | None | |
| Day 28 | 1 - | 1- | 1 | 1 | 1 | 2 - | +3 Very |
| | Clear | Flat | None | None | None | Mild | Much |
| | | | | | | | Improved |
| Day 56 | 1 - | 1 - | 3 - | 1 - | 3 - | 1 - | +3 Very |
| | Clear | Flat | Moderate | None | Moderate | None | Much |
| | | | | | | | Improved |
| Day 84 | 1 - | 1 - | 1 - | 1 - | 1 - | 1 - | +3 Very |
| | Clear | Flat | None | None | None | None | Much |
| | | | | | | | Improved |

TABLE 15
Patient Assessments at Clinical Visits

| | PGI-S | PGI-C | Blood Levels |
|---|---|---|---|
| Baseline | Mild | N/A | <2 ng/ml |
| Day 28 | Mild | +2 Much Improvement | <2 ng/ml |
| Day 56 | Mild | +2 Much Improvement | <2 ng/ml |
| Day 84 | Mild | +3 Very Much Improved | <2 ng/ml |

TABLE 16
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | None | None | N/A | N/A | 1 | 0/14 | 0/14 | 5.6 |
| Day 28 | None | None | Much | Minimal | 5 | 3/14 | 1/14 | 6.1 |
| | | | Improvement | Improvement | | | | |
| Day 56 | None | None | Much | Much | 3 | 0/13 | 0/13 | 3.8 |
| | | | Improvement | Improvement | | | | |
| Day 84 | None | None | Much | Much | 3.5 | 0/14 | 0/14 | 2.2 |
| | | | Improvement | Improvement | | | | |

### Example 8: Subject 6 Results

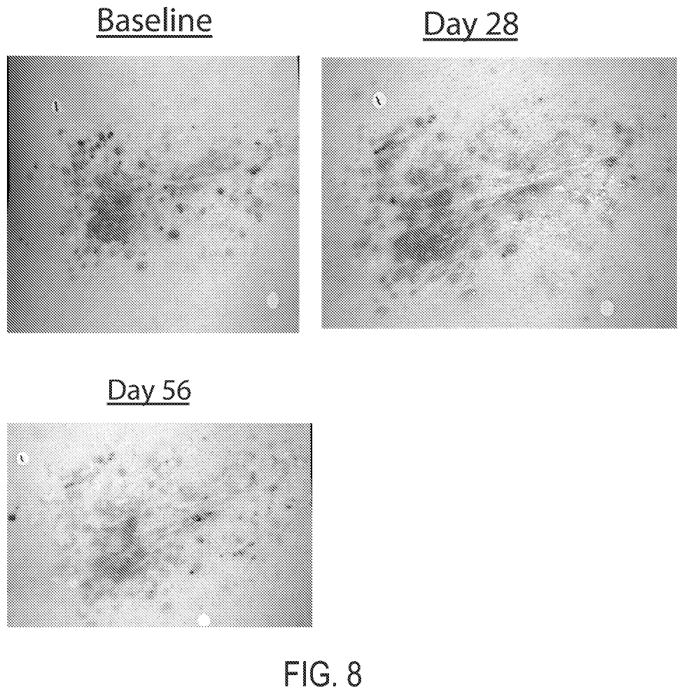

Subject 6 was enrolled in the study with moderate microcystic LM. FIG. 8 shows baseline of microcystic LM evaluation and day 28 and day 56 evaluations following treatment. The clinician assessments at clinical visits (Table 17), patient assessments at clinical visits (Table 18) and patient daily or weekly assessments (Table 19) are shown below.

TABLE 17
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 3 - | 3 - | 1 - | 2 - | 2 - | 2 - | N/A |
| | Moderate | Moderate | None | Mild | Mild | Mild | |
| Day 28 | 3 - | 3 - | 1 - | 2 - | 2 - | 1 - | +2 Much |
| | Moderate | Moderate | None | Mild | Mild | None | Improved |
| Day 56 | | | | | | | |

TABLE 18
Patient Assessments at Clinical Visits

| | PGI-S | PGI-C | Blood Levels |
|---|---|---|---|
| Baseline | Mild | N/A | <2 ng/ml |
| Day 28 | Moderate | +1 Minimal Improvement | <2 ng/ml |
| Day 56 | | | <2 ng/ml |

TABLE 19
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | None | Mild | | | 1.5 | 0/13 | 13/13 | 1.1 |
| Day 28 | Mild | None | Much | No | 4.5 | 6/12 | 12/12 | 4.3 |
| | | | worse, - No | change | | | | |
| | | | change | | | | | |
| Day 56 | None | None | Much | No | 0 | 0/7 | 2/7 | 1.0 |
| | | | Improvement | Change | | | | |

### Example 9: Subject 7 Results

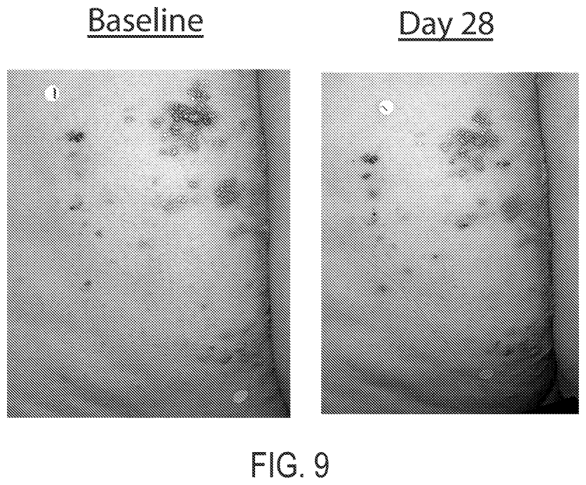

Subject 7 was enrolled in the study with moderate microcystic LM. FIG. 9 shows baseline microcystic LM evaluation and day 28 evaluation following treatment. The clinician assessments at clinical visits (Table 20), patient assessments at clinical visits (Table 21) and patient daily or weekly assessments (Table 22) are shown below.

TABLE 20
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 3 - | 4 - | 2 - | 2 - | 2 - | 3 - | N/A |
| | Moderate | Severe | Mild | Mild | Mild | Moderate | |
| Day 28 | 2- | 2 - | 1 - | 1 - | 1 - | 2 - | +1 |
| | | Mild | None | None | None | Mild | |
| Day 56 | | | | | | | |

TABLE 21
Patient Assessments at Clinical Visits

| | PGI-S | PGI-C | Blood Levels |
|---|---|---|---|
| Baseline | Moderate | N/A | <2 ng/ml |
| Day 28 | Moderate | +1 Minimal Improvement | <2 ng/ml |
| Day 56 | | | |

TABLE 22
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | None | None | N/A | N/A | 1 | 0/14 | 0/14 | 5.6 |
| Day 28 | Mild | Mild | No | No | 2 | 4/10 | 8/10 | 2.4 |
| | | | Change | Change | | | | |
| Day 56 | | | | | | | | |

### Example 10—Subject 8 Results

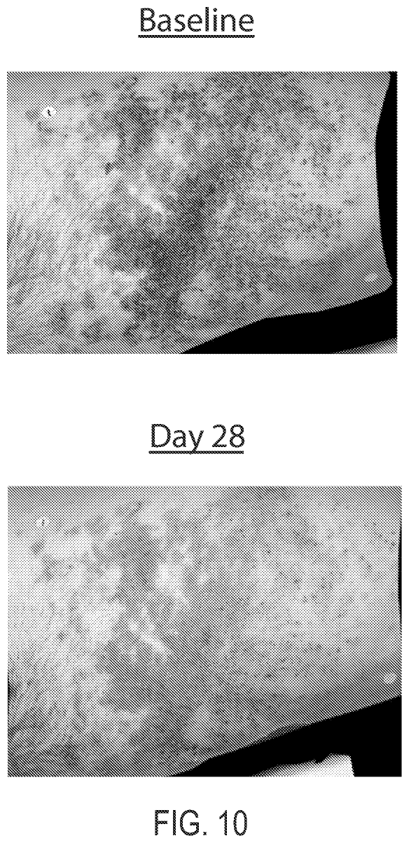

Subject 8 was enrolled in the study with severe microcystic LM. FIG. 10 shows baseline microcystic LM evaluation and day 28 evaluation following treatment. The clinician assessments at clinical visits (Table 23), patient assessments at clinical visits (Table 24) and patient daily or weekly assessments (Table 25) are shown below.

TABLE 23
Clinician Assessments at Clinical Visits

| | GI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 4 - | 4 - | 4 - | 4 - | 4 - | 4 - | N/A |
| | Severe | Severe | Severe | Severe | Severe | Severe | |
| Day 28 | 2 - | 2 - | 2 - | 1 - | 2 - | 2 - | +2 Much |
| | Mild | Mild | Mild | None | Mild | Mild | Improved |
| Day 56 | | | | | | | |

TABLE 24
Patient Assessments at Clinical Visits

| | PGI-S | PGI-C | Blood Levels |
|---|---|---|---|
| Baseline | Moderate | N/A | <2 ng/ml |
| Day 28 | Mild | +1 Minimal Improvement | <2 ng/ml |
| Day 56 | | | |

TABLE 25
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | Mild, | None | N/A | N/A | 4.5 | 3/14 | 14/14 | 1.2 |
| | Moderate | | | | | | | |
| Day 28 | | | | | | | | |
| Day 56 | | | | | | | | |

### Example 11—Subject 9 Results

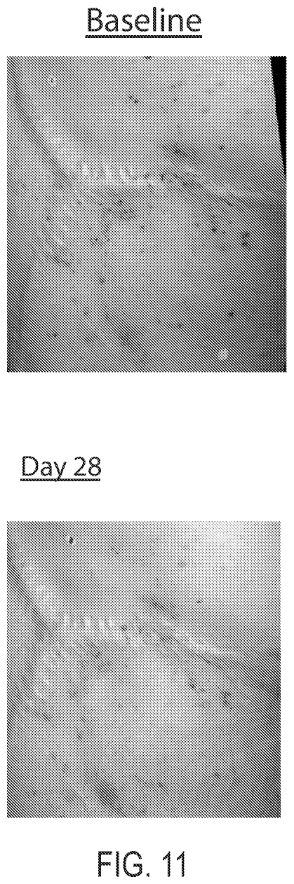

Subject 9 was enrolled in the study with moderate microcystic LM. FIG. 11 shows baseline microcystic LM evaluation and day 28 evaluation following treatment. The clinician assessments at clinical visits (Table 26), patient assessments at clinical visits (Table 27) and patient daily or weekly assessments (Table 28) are shown below.

TABLE 26
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 3 - | 3 - | 2 - | 1 - | 2 - | 2 - | N/A |
| | Moderate | Moderate | Mild | None | Mild | Mild | |
| Day 28 | 2 - | 2 - | 1 - | 1 - | 1 - | 1 - | +2 Much |
| | Mild | Mild | None | None | None | None | Improvement |
| Day 56 | | | | | | | |

TABLE 27
Patient Assessments at Clinical Visits

| | PGI-S | PGI-C | Blood Levels |
|---|---|---|---|
| Baseline | Moderate | N/A | <2 ng/ml |
| Day 28 | Mild | +1 Minimal Improvement | |
| Day 56 | | | |

TABLE 28
Patient Daily or Weekly Assessments

| | | | | | | | | Average |
|---|---|---|---|---|---|---|---|---|
| | PGI-S | PGI-S | PGI-C | PGI-C | Crusting | Days | Days | Pain |
| | Leaking | Bleeding | Leaking | Bleeding | Severity | Bleeding | Leaking | (14 days) |
| Baseline | Mild | Mild | N/A | N/A | 4 | 0/9 | 2/9 | 1.0 |
| Day 28 | | | | | | | | |
| Day 56 | | | | | | | | |

### Example 12—Subject 10 Results

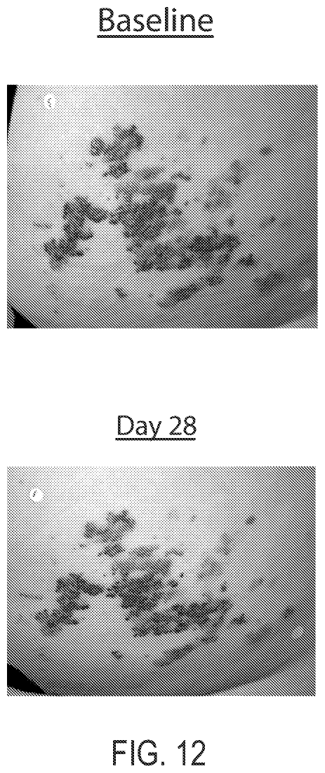

Subject 10 was enrolled in the study with severe microcystic LM. FIG. 12 shows baseline microcystic LM evaluation and day 28 evaluation following treatment. The clinician assessments at clinical visits (Table 29) and patient assessments at clinical visits (Table 30) are shown below.

TABLE 29
Clinician Assessments at Clinical Visits

| | CGI-S | CGI-S | CGI-S | CGI-S | CGI-S | CGI-S | |
|---|---|---|---|---|---|---|---|
| | Overall | Height | Leaking | Bleeding | Erythema | Hyperkeratosis | CGI-C |
| Baseline | 4 - | 4 - | 3 - | 4 - | 4 - | 3 - | N/A |
| | Severe | Severe | Moderate | Severe | Severe | Moderate | |
| Day 28 | 2 - | 2 - | 1 - | 1 - | 1 - | 2 - | +1 Minimal |
| | Mild | Mild | None | None | None | Mild | Improvement |
| Day 56 | | | | | | | |

TABLE 30
Patient Assessments at Clinical Visits

| | PGI-S | PGI-C | Blood Levels |
|---|---|---|---|
| Baseline | Severe | N/A | <2 ng/ml |
| Day 28 | | +1 Minimal Improvement | <2 ng/ml |
| Day 56 | | | |

### Example 10—Safety

Treatment related adverse events are shown in Table 23. One subject reported mild TRAEs including itching, tender nodule and tingling sensation. Two subjects reported moderate TRAEs including pain at application site, burning sensation, itching, nausea and redness. There were no clinically significant lab or vital sign abnormalities.

TABLE 31
Patient Reported TRAEs

| Treatment Related | | | | |
|---|---|---|---|---|
| Adverse Event | | | | |
| (N = 12) | Mild | Moderate | Severe | Total |
| Participants with Any | 1 (8.3%) | 2 (16.7%) | 0 | 3 (25%) |
| TRAE | | | | |

Number of Participants by Maximum Severity of each TRAE

| | | | | |
|---|---|---|---|---|
| Pain at Application | 0 | 1 (8.3%) | 0 | 1 (8.3%) |
| Site | | | | |
| Burning Sensation | 0 | 1 (8.3%) | 0 | 1 (8.3%) |
| Itching | 1 (8.3%) | 1 (8.3%) | 0 | 2 (16.7%) |
| Tender Nodule | 1 (8.3%) | 0 | 0 | 1 (8.3%) |
| Tingling Sensation | 1 (8.3%) | 0 | 0 | 1 (8.3%) |
| Nausea | 0 | 1 (8.3%) | 0 | 1 (8.3%) |
| Redness | 0 | 1 (8.3%) | 0 | 1 (8.3%) |

Although the invention has been described with reference to the above examples it will be understood that modifications and variations are encompassed within the spirit and scope of the invention. Accordingly, the invention is limited only by the following claims.

1. A method of treating microcystic lymphatic malformation in a subject in need thereof comprising topically administering once daily to the microcystic lymphatic malformation skin lesions of the subject a therapeutically effective amount of an anhydrous rapamycin gel composition wherein the microcystic lymphatic malformation is treated.

2. The method of claim 1, wherein the microcystic lymphatic malformation skin lesion is greater than about 25 cm².

3. The method of claim 1, wherein a symptom related to the microcystic lymphatic malformation skin lesion occurs at least 1-2 days per week.

4. The method of claim 1, wherein treating microcystic lymphatic malformation in a subject is improving a symptom of the microcystic lymphatic malformation skin lesion selected from the group consisting of leaking (lymphorrhea), bleeding, redness, crusting, hyperkeratosis, elevated plaques, and combinations thereof.

5. The method of claim 1, wherein the anhydrous rapamycin gel composition comprises about 0.1% to about 6% of rapamycin, about 80% to about 99% of one or more solvents, a gelling agent, and an antioxidant.

6. The method of claim 1, wherein the anhydrous rapamycin gel composition comprises about 0.1% to about 6% of rapamycin, about 80% to about 99% of one or more solvents, about 0.1% to about 5% of a gelling agent, and about 0.001% to about 1% of an antioxidant.

7. The method of claim 1, wherein the anhydrous rapamycin gel composition is administered once daily for at least 12 weeks.

8. The method of claim 5, wherein the one or more solvents comprises diisopropyl adipate, glycerol, PEG, and isopropyl alcohol.

9. The method of claim 1, wherein the anhydrous rapamycin gel composition comprises:
about 3.9% of rapamycin,
about 15% isopropyl alcohol,
about 54.9% polyethylene glycol 400,
about 15% diisopropyl adipate,
about 10% glycerol,
about 0.75% hydroxypropyl cellulose,
about 0.05% propyl gallate,
about 0.02% ascorbyl palmitate,
about 0.002% alpha-tocopherol, and
citric acid.

10. The method of claim 1, wherein administering comprises applying the anhydrous rapamycin gel composition as a thin layer to the subject's skin.

11. The method of claim 1, wherein the subject shows improvement in a score on one or more of assessments selected from the group consisting of Patient Global Impression of Change (PGI-C), Patient Global Impression of Severity (PGI-S), Patient Global Impression of Severity (PGI-SL) for leaking, Patient Global Impression of Severity (PGI-SB) for bleeding, Clinician Global Impression of Change (CGI-C), Clinician Global Impression of Severity (CGI-S), and combinations thereof.

12. The method of claim 1, wherein the subject shows improvement in a score on the dermatology life quality index (DLQI).

13. The method of claim 1, wherein the subject experiences a decrease in the number of days in which lesion leaking is reported.

14. The method of claim 1, wherein the subject experiences a decrease in the number of days in which lesion bleeding is reported.

15. The method of claim 1, wherein one or more of lesion size lesion thickness, lesion height, or lesion number is reduced.

16. The method of claim 1, wherein the severity of lesion pain, lesion crusting or lesion redness based on the Microcystic LM Symptom Severity (SS) Scale is reduced.

17. The method of claim 1, wherein field radiation (XRT) administered to the mycrocystic lymphatic malformation skin lesion is not necessary.

18. The method of claim 1, wherein topical administration of the anhydrous rapamycin gel composition to the skin of a subject achieves a maximum epidermis concentration of rapamycin in the epidermis of the subject of about 120-990 micromolar rapamycin.

19. The method of claim 1, wherein topical administration of the anhydrous rapamycin gel composition to the skin of a subject achieves a maximum dermis concentration of rapamycin in the dermis of the subject of about 50-200 micromolar rapamycin.
